(19) United States
(12) Patent Application Publication
(10) Pub. No.: US 2024/0216481 A1
(43) Pub. Date: Jul. 4, 2024

(54) COMPOSITION FOR TREATING OF FACIAL DYSMORPHISM IN MUCOPOLYSACCHARIDOSIS

(71) Applicants: GREEN CROSS CORPORATION, Yongin (KR); Dong Kyu JIN, Seoul (KR)

(72) Inventor: Dong Kyu JIN, Seoul (KR)

(73) Assignees: GREEN CROSS CORPORATION, Yongin (KR); Dong Kyu JIN, Seoul (KR)

(21) Appl. No.: 18/541,328

(22) Filed: Dec. 15, 2023

Related U.S. Application Data
(62) Division of application No. 18/148,019, filed on Dec. 29, 2022, now abandoned.

(51) Int. Cl. A61K 38/46; A61K 9/00; A61K 31/728; A61P 3/00

(52) U.S. Cl. CPC A61K 38/465; A61K 9/0019; A61K 31/728; A61P 3/00; C12Y 301/06013

(57) ABSTRACT

A composition and a use thereof in treating facial dysmorphism in mucopolysaccharidosis are disclosed. The composition is obtained by subcutaneously injecting a composition into the face for facial subcutaneous injection formulation including a lysosomal enzyme and hyaluronic acid into a subject, thereby effectively improving facial dysmorphism in mucopolysaccharidosis

### CROSS REFERENCE TO RELATED APPLICATIONS

This application is a Division of U.S. application Ser. No. 18/148,019, filed Dec. 29, 2022, the content of which is incorporated herein by reference in its entirety.

### INCORPORATION BY REFERENCE OF SEQUENCE LISTING

The content of the electronically submitted sequence listing, file name: Q294397_SEQ_LIST_ST26_AS_FILED.xml; size: 1.5 kilobytes; date of creation: Dec. 27, 2022; and date of modification: Dec. 14, 2023, filed herewith, is incorporated herein by reference in its entirety.

### BACKGROUND

### 1. Field

The present invention relates to a composition for treating of facial dysmorphism in mucopolysaccharidosis.

### 2. Description of the Related Art

Mucopolysaccharidosis is a genetic disorder caused by a lack of lysosomal enzymes necessary for the breakdown of glycosaminoglycans. As glycosaminoglycans accumulate in lysosomes of cells and are excessively excreted in urine, physical and mental degeneration gradually progresses. Severe symptoms can lead to premature death and various clinical manifestations. Clinical types are classified into types 1 to 7 depending on which enzyme is deficient.

Mucopolysaccharidosis shows various symptoms depending on the type or degree of deficiency of the enzyme. It occurs when the metabolism of dermatan sulfate, eparan sulfate, and keratan sulfate, which are included in glycosaminoglycans, is impaired and accumulates in each organ of the body. Most patients show severe intellectual disability and physical abnormalities due to accumulation of mucopolysaccharide, and in severe cases die early.

These diseases generally show common clinical features. Typical examples include chronic progression, accumulation in multiple organs, organ hypertrophy, bone and facial abnormalities. Hearing impairment, visual impairment, respiratory failure, cardiac dysfunction, and joint abnormalities are frequently observed. All of these diseases, except for type 2, are inherited in an autosomal recessive manner.

Regarding such Mucopolysaccharidosis, enzyme replacement therapy for supplementing the deficient enzyme is being implemented as a treatment, thereby improving physical signs and symptoms. However, there is a problem that patients continue to show facial dysmorphism despite such enzyme replacement therapy.

### SUMMARY

It is an object of the present invention to provide a pharmaceutical composition for treating of facial dysmorphism.

It is an object of the present invention to provide a method for treating of facial dysmorphism.

To achieve the above object, the following technical solutions are adopted in the present invention.
1. A composition for treating of facial dysmorphism in mucopolysaccharidosis, comprising a lysosomal enzyme and hyaluronic acid, which is a facial subcutaneous injection formulation.
2. The composition according to the above 1, wherein the lysosomal enzyme is iduronate-2-sulfatase (IDS).
3. The composition according to the above 2, wherein the iduronate-2-sulfatase is composed of the amino acid sequence of SEQ ID NO: 1 or 2.
4. The composition according to the above 1, wherein the lysosomal enzyme is included in a concentration of 0.5 mg/kg to 20 mg/kg.
5. The composition according to the above 1, wherein the hyaluronic acid has a molecular weight of 1000 to 5000 kDa.
6. The composition according to the above 1, wherein the hyaluronic acid is included at a concentration of 1 mg/ml to 15 mg/ml.
7. The composition according to the above 1, wherein the mucopolysaccharidosis is type 1 or type 2 mucopolysaccharidosis.
8. The composition according to the above 1, wherein the mucopolysaccharidosis is type 2 mucopolysaccharidosis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and other advantages of the present invention will be more clearly understood from the following detailed description taken in conjunction with the accompanying drawings, in which:

FIG. 1. Measured viscosity graph of the combination drugs of idursulfase beta (IDS-β) and hyaluronic acid (HA). C1: IDS-β 1.5 mg/mL and HA 5 mg/mL, C2: IDS-β 3 mg/mL and HA 5 mg/mL, C3: IDS-β 1.5 mg/mL and HA 7 mg/mL, C4: IDS-β 3 mg/mL and HA 7 mg/mL, C5: IDS-β 1.5 mg/mL and HA 9 mg/mL, C6: IDS-β 3 mg/mL and HA 9 mg/mL, C7; IDS-β 1.5 mg/mL and HA 12 mg/mL, C8: IDS-β 3 mg/mL and HA 12 mg/mL.

FIG. 2. In vitro release test graph of the combinations of idursulfase beta (IDS-β) and hyaluronic acid (HA). C1: IDS-β 1.5 mg/mL and HA 5 mg/mL, C2: IDS-β 3 mg/mL and HA 5 mg/mL, C3: IDS-β 1.5 mg/mL and HA 7 mg/mL, C4: IDS-β 3 mg/mL and HA 7 mg/mL, C5: IDS-β 1.5 mg/mL and HA 9 mg/mL, C6; IDS-β 3 mg/mL and HA 9 mg/mL, C7; IDS-β 1.5 mg/mL and HA 12 mg/mL, C8: IDS-β 3 mg/mL and HA 12 mg/mL.

FIG. 3. The structures of vessels and nerves in the mouse face and the SC injection procedure. The left figure shows the structures of blood vessels in the mouse face and the middle figure shows the nerves. The figure on the right shows the IACUC Standard Procedure.

FIG. 4. Description of parameters for 3D scanning image analysis. (A) Facial Depth, (B) Facial Depth: Central, Right, Left, (C) Facial volume, (D) Eye distance, (E) Snout length, and (F) Snout circumference.

FIG. 5. Schematic diagram of the study.

FIG. 6. Facial morphology of the A mouse group with facial subcutaneous injections twice a month: (a) snout angle view and (b) side view photography. SC: facial subcutaneous injection, IDS: iduronate-2-sulfatase, KO; knockout, IDS KO; non-treated MPS II mice.

FIG. 7. Facial morphology of the B mouse group with facial subcutaneous injection and intravenous infusion: (a) snout angle view and (b) side view photography. IV: intravenous infusion, SC: subcutaneous facial injection, IDS: iduronate-2-sulfatase, KO; knockout, IDS KO; non-treated MPS II mice.

FIG. 8. The B mouse group with facial subcutaneous injection or intravenous infusion: (a) snout angle view and (b) side view photography. IV: intravenous infusion, SC: facial subcutaneous injection.

FIG. 9. Histopathological changes in the skin of MPS II mice observed two weeks after the last dose and wild type (WT) mice of the same age. (A) H&E staining. Representative photomicrographs of the facial skin sections are shown. The second column is a high-power view of the first column photomicrographs. Red arrows indicate cytoplasmic vacuoles in hair follicle cells and blue arrows indicate cytoplasmic vacuoles in the epidermal keratinocytes. (B) AB PAS staining. Representative photomicrographs of sections from the facial skin sections are shown and blue stains indicate positive signals for glycosaminoglycans. The black scale bars are 200 μm and the white scale bars are 60 μm. IV: intravenous infusion, SC: facial subcutaneous injection.

FIG. 10. MPS II mice (black arrow) at 16 weeks of age (body weight: 30.67 g), compared to a WT mouse (plaid pattern arrow) at 16 weeks of age (body weight: 30.70 g). (a) MPS II mice showed progressive worsening of coarse fur, distorted faces, and (b, c) broadened snouts as a result of sclerosis and enlargement of the skull bones. (d) The hair of the MPS II mice is thicker and shows more alopecia than the WT mice. The fore (e) and hind feet (f) are broader and stiffer with the paw curled underneath.

FIG. 11. 3D scanning enables the concurrent 3D analyses of the faces of MPS II mice compared to the WT mice group at 18 weeks of age. (Left) The mean surface shape of each group is shown in the left column and the second-fifth columns show comparisons in various directions in colour. 3D analysis is performed laterally in the x-axis, vertically in the y-axis, and depth-wise in the z-axis direction. A value with no difference between the two groups is green in colour, positive difference values are red, and negative difference values are purple.

FIG. 12. Differences in facial dysmorphology between the WT and MPS II mouse groups without treatment. Transverse bars indicate the median values and dots indicate individual values. Each group described at the bottom of the drawing is shown in the drawing in order from the right.

FIG. 13. Results of concurrent 3D analyses of faces of non-treated MPS II mice compared to the intravenous-treated group at 18 weeks of age. (Left) The mean surface shape of each group is shown in the left column and the second-fifth columns show comparisons between various directions in colour. 3D analysis was performed laterally in the x-axis, vertically in the y-axis, and depth-wise in the z-axis direction. A value with no difference between the two groups is green, a positive difference value is red, and a negative difference value is purple.

FIG. 14. Differences in facial dysmorphology between intravenous treatment and non-treatment IDS KO, WT, or facial subcutaneous-treated MPS II groups. Transverse bars indicate median values; dots indicate individual values. Each group described at the bottom of the drawing is shown in the drawing in order from the left.

FIG. 15. Results of concurrent 3D analyses of the faces of non-treated MPS II mice compared to subcutaneous-treated IDS KO mice at 18 weeks of age. (Left) The mean surface shape of each group is shown in the left column and the second-fifth columns show comparisons in various directions in colour. 3D analyses are performed laterally in the x-axis, vertically in the y-axis, and depth-wise in the z-axis directions, respectively. A value with no difference between the two groups is green, positive differences are red, and negative differences are purple.

FIG. 16. Differences in facial dysmorphology between facial subcutaneous injection and non-treatment MPS II groups. The following parameters are shown: (A) facial volume, (B) facial depth, (C) snout circumference, (D) snout length, (E) eye distance, (F) sulfatedGAG, and (G) dermis thickness. Each group of 0.5, 2.5, 5.0, and 10 mg/kg according to the facial subcutaneous injection dose was indicated in order from left to right. Transverse bars indicate median values: dots indicate individual values.

FIG. 17. Results of concurrent 3D analyses of faces of non-treated MPS II mice compared to IV-and-SC-treated mice at 18 weeks of age. (Left) The mean surface shape of each group is shown in the left column, and the second to fifth columns show comparisons in various directions in colour. 3D analyses are performed for the lateral direction, vertical direction, and depth in the x-axis, y-axis, and z-axis, respectively. A value with no difference between the two groups is depicted in green, a positive difference value is depicted in red, and a negative difference value is depicted in purple.

FIG. 18. Differences in facial dysmorphology of IV-and-SC-treated vs. non-treated MPS II groups. The following parameters were used: (A) facial volume, (B) facial depth, (C) snout circumference, (D) snout length, (E) eye distance, (F) sulfated GAG, and (G) dermis thickness. Each group of 0.5, 2.5, 5.0, and 10 mg/kg according to the SC facial injection dose is indicated in order from left to right. Transverse bars indicate median values: dots indicate individual values.

FIG. 19. Results of concurrent 3D analyses of the faces of IV-treated MPS II mice compared to IV-and-SC-treated mice at 18 weeks of age. (Left) The mean surface shape of each group is shown in the left column, and the second to fifth columns show comparisons in various directions in colour. 3D analyses are performed for the lateral direction, vertical direction, and depth in the x-axis, y-axis, and z-axis, respectively. A value with no difference between the two groups is depicted in green, a positive difference value is depicted in red, and a negative difference value is depicted in purple.

FIG. 20. Differences in facial dysmorphology of IV-treated vs. IV-and-SC-treated groups. The following parameters were used: (A) facial volume, (B) facial depth, (C) snout circumference, (D) snout length, (E) eye distance, (F) sulfated GAG, and (G) dermis thickness. Each group of 0.5, 2.5, 5.0, and 10 mg/kg according to the SC facial injection dose is indicated in order from left to right. Transverse bars indicate median values; dots indicate individual values.

FIG. 21. Results of concurrent 3D analyses of the faces of SC-treated MPS II mice compared to IV-and-SC-treated mice at 18 weeks of age. (Left) The mean surface shape of each group is shown in the left column, and the second to fifth columns show comparisons in various directions in colour. 3D analyses are performed laterally in the x-axis, vertically in the y-axis, and depth-wise in the z-axis directions, respectively. A value with no difference between the two groups is green, positive differences are red, and negative differences are purple.

FIG. 22. Differences in facial dysmorphology between the SC-treated and IV-and-SC-treated groups. Transverse bars indicate median values: dots indicate individual values. In the figure, the SC treatment group is indicated on the left, and the IV and SC treatment groups are indicated on the right.

FIG. 23. A box plot for body weight change from 4 to 12 weeks of age for groups A and B (Mann-Whitney test: p<0.001).

FIG. 24. The mean body weight change by facial SC injection dose of mice in each subgroup in groups A and B from 4 to 12 weeks of age.

FIGS. 25A and 25B. Comparison of weight gain (%) for the SC-treated group (A1, A2, A3, A4), and IV-and-SC-treated group (B1, B2, B3, B4) for 2 months. In FIG. 25A, transverse bars indicate median values: dots indicate individual values. In FIG. 25B, box plot shows change in body weight gain (%). P-values calculated using the Mann-Whitney test were <0.001.

FIG. 26. Comparison of weight gain (%) for 2 months between groups with the facial SC injection procedure (+: A1, A2, A3, A4, B1, B2, B3, B4, B5, B6) and groups without the facial SC injection procedure (−: A5, B7). The p-value calculated using the Mann-Whitney test was 0.211.

FIGS. 27A and 27B. Correlation of 3D image data and pathologic results. FIG. 27A shows correlation of facial volume and sulfated GAG. FIG. 27B shows correlation of facial volume and dermis thickness.

### DETAILED DESCRIPTION

Hereinafter, the present invention will be described in detail. Unless otherwise specifically defined, all terms in the present specification would have the same meanings as general meanings of the corresponding terms understood by persons having common knowledge to which the present invention pertains (“those skilled in the art”), and if the general meanings conflict with the meanings of the terms used herein, the meanings used in the present specification take precedence.

The present invention relates to a pharmaceutical composition for treating of facial dysmorphism in mucopolysaccharidosis.

The composition for treating facial dysmorphism in mucopolysaccharidosis of the present invention contains a lysosomal enzyme and hyaluronic acid, and is a subcutaneous injection formulation for the face.

Mucopolysaccharidosis is a genetic disorder caused by a lack of lysosomal enzymes necessary for the breakdown of glycosaminoglycans. As glycosaminoglycans accumulate in lysosomes of cells and are excessively excreted in urine, physical and mental degeneration gradually progresses. Severe symptoms can lead to premature death and various clinical manifestations. Clinical types are classified into types 1 to 7 depending on which enzyme is deficient.

Mucopolysaccharidosis shows clinical common features such as chronic progressiveness, accumulation in multiple organs, organ hypertrophy, and bone and facial abnormalities (facial dysmorphism).

In the case of facial dysmorphism, it appears in the form of a large head with a protruding front forehead, a low and wide nose, thick lips, and a large tongue, etc.

The composition of the present invention exhibits a therapeutic effect on such facial dysmorphism in mucopolysaccharidosis.

In the present invention, the facial dysmorphism to be treated in the present invention is a facial dysmorphism in mucopolysaccharidosis, which may be one of types 1 to 7, and may be type 1 or type 2, specifically type 2.

In the present invention, lysosomal enzymes can be deficient lysosomal enzymes in each type of mucopolysaccharidosis (types 1 to 7). For example, if mucopolysaccharidosis is type 1, it may be alpha-L-iduronidase, if it is type 2, it may be iduronate-2-sulfatase, if it is type 3, it may be heparan N-sulfatase, N-acetyl-alpha-D-glucosaminidase, acetyl-CoA:alpha-glucosaminide-N-acetyltransferase or N-acetyl-alpha-D-glucosamine-6-sulfatase, if it is type 4, it may be N-acetylgalactosamine-6 sulfatase, and beta-galactosidase, if it is type-6, it may be arylsulfatase B, if it is type 7, it may be beta-glucuronidase.

In the present invention, the lysosomal enzyme may be iduronate-2-sulfatase (IDS-β (idursulfatase beta, International Nonproprietary Name for iduronate-2-sulfatase). Any known in the art may be used, for example, those comprising the amino acid sequence of SEQ ID NO: 1 or 2 can be used. Alternatively, commercially available drugs can be used, for example, Hunterase (Green Cross, Korea) can be used. HUNTERASER is a commercially available drug of Green Cross, Yongin, Korea, which contains the recombinant human iduronate-2-sulfatase (idursulfase beta, IDS-β).

In the present invention, the lysosomal enzyme may be included, for example, in a concentration of 0.5 mg/kg to 30 mg/kg, 1 mg/kg to 20 mg/kg, 5 mg/kg to 20 mg/kg, 10 mg/kg to 20 mg/kg.

Hyaluronic acid (HA) is a natural substance having biocompatibility that is abundantly present in animal skin, joint fluid, and cartilage. It is a component constituting the matrix in articular cartilage and is a kind of mucopolysaccharide involved in making proteoglycan. It is a glycoprotein complex of N-acetyl-D-glucosamine and D-Glucuronic Acid linked by 1-4 glycosidic linkages. Hyaluronic acid can be combined with water to exist in a gel state.

In the present invention, Hyaluronic acid may have, for example, a molecular weight of 1000 kDa to 5000 kDa or 2000 kDa to 4000 kDa.

In the present invention, hyaluronic acid may be cross-linked through a physical method such as ultraviolet rays, radiation, electron beams, or a chemical method using 1,4-butanediol diglycidyl ether (BDDE).

In the present invention, hyaluronic acid may be included as a pharmaceutically acceptable salt. Pharmaceutically acceptable salts of hyaluronic acid include, for example, both inorganic salts such as sodium hyaluronate, magnesium hyaluronate, zinc hyaluronate and cobalt hyaluronate, and organic salts such as tetrabutylammonium hyaluronate. In some cases, a combination of at least two of these may be used.

In the present invention, hyaluronic acid may be included, for example, in a concentration of 1 mg/ml to 20 mg/ml, 1 mg/ml to 18 mg/ml, 3 mg/ml to 18 mg/ml, 5 mg/ml to 18 mg/ml, 5 mg/ml to 15 mg/ml, 8 mg/ml to 15 mg/ml.

In addition, the present invention relates to a method for treating facial dysmorphism in mucopolysaccharidosis.

The method of the present invention comprises subcutaneous injection of a composition comprising a lysosomal enzyme and hyaluronic acid to the face of a subject in need thereof.

In the present invention, the subject may be an animal that has mucopolysaccharidosis and exhibits facial dysmorphism. The subject may be a mammal, including a human, or may be a human.

In the present invention, mucopolysaccharidosis may be one of types 1 to 7, and may be type 1 or type 2, specifically type 2.

In the case of facial dysmorphism, symptoms may include a large head with a protruding front forehead, a low and wide nose, thick lips, and a large tongue.

In the present invention, the composition comprising a lysosomal enzyme and hyaluronic acid may include all possible combinations within the ranges exemplified above.

In the present invention, subcutaneous injection can be performed according to methods known in the art. For example, needles of 16 gauge to 32 gauge, 25 gauge to 35 gauge, and 25 gauge to 31 gauge may be used.

In the present invention, injections can be performed at suitable intervals and for a suitable period of time. The interval may be, for example, once to three times a week. The period may be performed until the facial anomaly is improved to a desired level. For example, it may be 1 day to several years, 1 day to 1 year, 1 day to 6 months, 1 month to 6 months, 3 months to 6 months, etc., but is not limited thereto.

Hereinafter, the present invention will be described in detail with reference to the following examples.

### 1. Animals

### 1) Preparation of IDS Knockout (KO) Mice

All animal experiments were performed with the approval of the Institutional Animal Care and Use Committee, Laboratory Animal Research Center, Samsung Biomedical Research Institute Seoul, Korea (SBRI). Four-week-old male mice were used in this study. A previously study reported an IDS KO mice model was used as an MPS II animal model. Briefly, KO mice were prepared by replacing exons 2 and 3 of the IDS gene with a neomycin resistance gene. Inbreeding was used to produce animals. A six-to-eight-week-old WT brother-heterozygotic-sister mating system was used. Carrier females were bred with male mice on a B6/129 background, producing heterogeneous females, hemizygous male KO mice, WT males, and female littermates. The genotypes of all mice were confirmed via polymerase chain reaction using DNA obtained from a tail snip. The C57BL/6 strain was used as WT control mice. After the determination of genotypes, the animals were allocated to groups by stratified randomization depending on random digits that were assigned to the mice.

### 2) Maintenance Conditions

Mice need to be housed under specific environmental parameters, otherwise they may experience stress. The Guide for the Care and Use of Laboratory Animals, 8th edition is an internationally accepted document that outlines what is appropriate. The mice were housed at an acceptable temperature range of 20-26° C. and the humidity was in the range 30-70%. There were 15-24 air changes per hour and illumination was 12 hours of artificial light per day (08:00-20:00). The lights were operated manually for any observations or examinations conducted outside this stated time period. The room was cleaned daily with disinfectant. Cages and bedding were exchanged at least once weekly and food containers and racks were exchanged at least once every 4 weeks with autoclaved replacements (121° C. for 30 minutes). The size of cages made of poly-sulfone material was 310 mm (diameter)×220 mm (width)×160 mm (height) and Aspen bedding (Tapvei) was placed in each cage. There were five mice per cage and enrichment toys were provided. In the laboratory setting, the mice were fed solid food (5053, LabDiet), which was available ad libitum for each animal. Water conforming to the water quality standards required by the Korea Waterworks Law was available ad libitum. The water was analysed four times per year by the Institute of Industrial Pollution Co., Ltd.

### 2. Dose Formulation and Analysis

### 1) Preparation of IV ERT

For ERT, recombinant human iduronate-2-sulfatase beta (Hunterase®), Green Cross Corp., Yongin, Korea) was used and administered to the IDS-ERT group in the form of IV injections. The IDS-β treatment is well-tolerated in Korean patients with MPS II. The dosage (0.5 mg/kg) of IDS-β was injected once per week into the lateral tail vein of the MPS II mice.

### 2) Preparation of the Combination Drug Consisting of IDS-β and Hyaluronic Acid

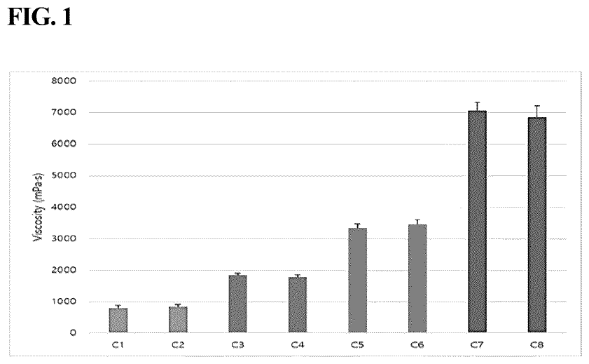

Hyaluronic acid (HA) of 3,000 kDa molecular weight was dissolved in a buffer to a 17 mg/mL concentration using a planetary mixer at 1,400 rpm/1,240 rpm for four hours. IDS-β was concentrated by centrifugation at 3,500 rpm for 15 minutes using a 10 kDa molecular weight cutoff (MWCO) centrifugal filter. The concentration of IDS-β was up to 16 mg/ml, which is eight times the conventional concentration. The concentrated IDS-β drug solution and HA solution were mixed to prepare the combination drugs of IDS-β and HA at the target concentration. The maximum concentration of HA was established as 12 mg/mL considering the injectable viscosity with a 29 G needle. Viscosity and protein concentration were analysed for various combination drugs using a rheometer and nanodrop (Table 1 and FIG. 1). The combination drug had a higher HA concentration and higher viscosity and the IDS-β concentration did not affect the combination drug's viscosity.

### 3) In Vitro Release Test for Measuring the Viscosity of the Combination Drugs

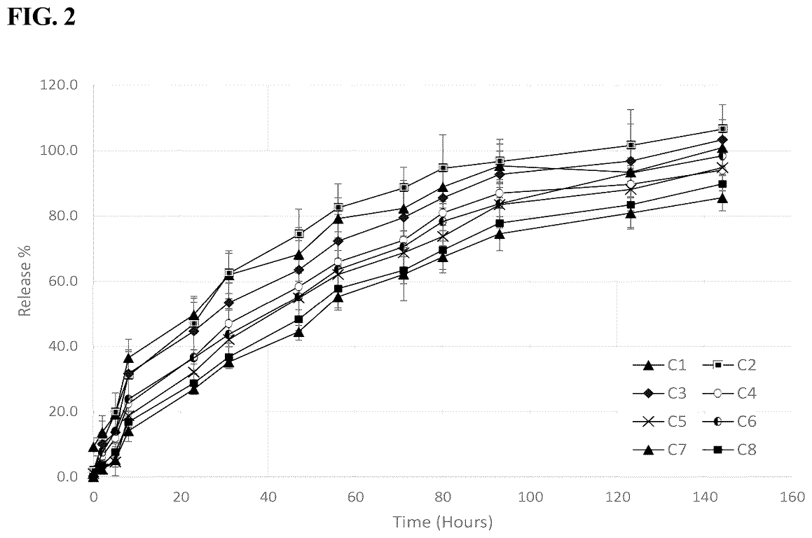

In vitro release tests were conducted using various concentrations of the combined drugs of IDS-β and HA. Each mixture (0.1 mL) was loaded into the bottom of a 5 mL tube with 2 mL of buffer. These tubes were input into a shaking incubator set to 37° C. At a pre-determined time-point, the upper solution was sampled and fresh buffer was added. The protein concentration was determined by Bradford assay. The in vitro release test results showed that the release rate could be controlled by HA concentration and the viscosity of the combination drug (FIG. 2). The final (140 hours) order in which IDS-β was released the most was C2, C3, C1, C6, C5, C4, C8, and C7.

TABLE 1
Measured viscosity of combination drug of IDS-β and HA

| | | | | Analysis results of combination drugs | |
|---|---|---|---|---|---|
| | Target concentration | | | Idursulfase beta | Viscosity |
| | Idursulfase | Hyaluronic | | concentration | mPa · s |
| | beta (IDS-β) | acid (HA) | | (Nanodrop) | (Rheometer) |
| C1 | 1.5 mg/mL | 5 | mg/mL | 1.6 mg/mL | 844.54 |
| | | | | 1.7 mg/mL | 710.61 |
| | | | | 1.6 mg/mL | 830.43 |
| C2 | 3.0 mg/mL | 5 | mg/mL | 3.0 mg/mL | 810.52 |
| | | | | 3.0 mg/mL | 780.3 |
| | | | | 3.0 mg/mL | 911.64 |
| C3 | 1.5 mg/mL | 7 | mg/mL | 1.5 mg/mL | 1,802.9 |
| | | | | 1.6 mg/mL | 1,778.6 |
| | | | | 1.7 mg/mL | 1,911.8 |
| C4 | 3.0 mg/mL | 7 | mg/mL | 3.1 mg/mL | 1,826.6 |
| | | | | 3.2 mg/mL | 1,699.2 |
| | | | | 3.2 mg/mL | 1,811.8 |
| C5 | 1.5 mg/mL | 9 | mg/mL | 1.6 mg/mL | 3,195.2 |
| | | | | 1.7 mg/mL | 3,432.7 |
| | | | | 1.6 mg/mL | 3,388.1 |
| C6 | 3.0 mg/mL | 9 | mg/mL | 3.1 mg/mL | 3,622 |
| | | | | 3.3 mg/mL | 3,378.8 |
| | | | | 3.3 mg/mL | 3,368.2 |
| C7 | 1.5 mg/mL | 12 | mg/mL | 1.5 mg/mL | 6,751.9 |
| | | | | 1.6 mg/mL | 7,160.9 |
| | | | | 1.6 mg/mL | 7,247.8 |
| C8 | 3.0 mg/mL | 12 | mg/mL | 3.1 mg/mL | 6,522.4 |
| | | | | 3.2 mg/mL | 6,780.3 |
| | | | | 3.2 mg/mL | 7,241.4 |

### 4) Justification for the Selection of HA Concentration Level

The release rate of IDS-β from the combination drug could be controlled by the HA concentration. A higher HA concentration showed an elongated release profile for IDS-β, which means that it had a better effect on the sustained release profile. The release time for 50% of IDS-β from the combination drug with 12 mg/mL HA was twice that of the 5 mg/mL HA combination drug. Therefore, the maximum concentration of HA (12 mg/mL) with injectable viscosity using a 29 G needle was selected as the therapeutic combination drug concentration for facial subcutaneous injection in this study.

### 5) Preparation of New Recombinant Protein Drugs for Positive Experimental Groups

The dose level of IDS-β for SC and IV injections was determined based on previous pharmacokinetic studies. The positive experimental groups consisted of 0.5, 2.5, 5, and 10 mg/kg IDS-β dose groups. The combination group of IDS-β and HA was prepared with an IDS-β concentration of 12 mg/ml. Then, 3.3 ml/kg of each dosage was subcutaneously injected into the mice. Individual doses were calculated based on the most recent bodyweight measurement.

### 6) Preparation of Vehicle for Facial SC Injection

For vehicle preparation, 8.8±0.2 g of sodium chloride was added to 900 mL of water for injection and mixed for 20-30 minutes, and water for the injection was added up to 1,000 mL final volume (Solution 1). Then, 2.5±0.1 g of Tween 20 was added to 40 mL of Solution 1 and mixed for 20-30 minutes, and Solution 1 was expanded to 50 mL (Solution 2). Approximately 0.05 g of Solution 2 was added to 40 mL of Solution 1 and mixed for 20-30 minutes, and Solution 1 was added to make 50 mL of 150 mM sodium chloride·0.05 mg/mL Tween 20 solution.

### 3. Procedures

### 1) Facial SC Injection

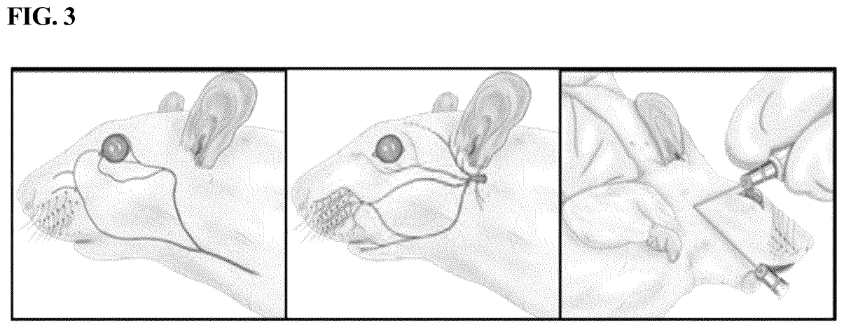

The mice were manually restrained with the non-dominant hand by grasping the loose skin over the shoulders and behind the ears. Then, the area surrounding both cheeks was wiped with 70% alcohol on a gauze sponge or swab. To inject while avoiding the blood vessels and nerve structures on the face of the mice, the central area under both cheeks was the target point (FIG. 3). The standard procedure for the submandibular blood collection in mice from the IACUC Standard Procedure was used as follows: measure a straight line from the bottom of the mandible to the caudal portion of the ear and from the top of the eye to the point of the shoulder (FIG. 3). If you puncture at the point where these two lines intersect, then you can collect a blood sample. Therefore, puncture the part in front of that point, as indicated by the arrow. The 29 G needle of an insulin syringe was inserted at a 45° angle to penetrate 1-2 mm into the subcutaneous tissue between the fat layer and the muscle layer below the facial skin surface, slightly float the needle to prevent deep penetration into the muscle layer. The total duration of administration was approximately 0.5-1 minute. Then, remove the needle and press the insertion site with forceps to prevent the injected drug from leaking out onto the skin. While injecting the drug, avoid the facial artery and vein, and be careful near the buccal branch of the facial nerve.

### 2) IV Injection

The mouse is placed in a restrainer. The mouse's tail is swabbed with 70% alcohol on a gauze sponge or swab. Insert the needle parallel to the tail vein penetrating 2-4 mm into the lumen while keeping the bevel of the needle face upward. Then, the solution is injected slowly and no resistance should be felt if the solution has been properly administered. When the intravenous administration is finished or the cannula is removed, the injection site must be pressed firmly with a swab or fingers to prevent backflow of the administered solution and/or blood.

4. Preparation of Tissue Extracts for s-GAG Analysis and Quantitative Analysis of s-GAG Accumulation

Facial skin specimens were fixed for 24 h in 10% neutral buffered formalin (NBF) and embedded in paraffin. Tissue extracts were prepared by homogenizing tissues in phosphate buffer saline (PBS) using a tissue homogenizer. Homogenates were centrifuged at 20,000×g for 30 min and supernatants were collected. The total protein concentration (mg/ml) was assayed using a bicinchoninic acid (BCA) assay (Pierce, Rockford, IL, USA). The GAG levels in tissue extracts were adjusted for protein concentration, which was determined using the BCA assay, and expressed as μg GAG per mg protein.

Tissue sections (4 μm thick) were stained with haematoxylin and eosin (H&E), and Alcian Blue (AB) with periodic acid and Schiff's solution (PAS) staining was done using the AB PAS Stain Kit (#ab245876; Abcam). The H&E staining of sections was performed in accordance with standard protocols. Images of each section were captured with a magnifier digital camera, using an Upright Microscope (DP80, Olympus, Japan) and saved as JPEG files. Image-Pro software (Media cybernetics, USA) was used as an image-analysis tool. Then, the epidermis thickness (μm), dermis thickness (μm), wrinkle depth (μm), and AB PAS positive area (%) were evaluated. The s-GAGs were measured using an Alcian blue-binding assay (Wieslab® s-GAG quantitative kit). The assay is used to detect s-GAG in biological samples such as synovial fluid, blood, and tissue extracts. The tetravalent cationic dye AB PAS is based on the ionic bonding with s-GAG, carboxyl groups, and phosphoric acid groups. Wieslab® is performed at a sufficiently low pH to neutralize all carboxylic and phosphoric acid groups and at a sufficiently large ionic strength to eliminate all ionic interactions other than those between AB PAS and s-GAG. Hyaluronan, a non-sGAG, does not react in this assay. There is no interference from proteins or nucleic acids in this method, in contrast to other dye binding methods.

### 5. 3D Scan Technology

The first ever 3-in-1 3D scanner, Drake, can digitize objects as small as a coin and as large as a tractor. The 3D scanning can be conducted well under laboratory room-lighting conditions, as can scanning in complete darkness. Furthermore, Drake is unique because it uses two projectors and a proprietary mathematical method to achieve unmatched results while scanning the most difficult objects. Especially, the Drake Mini has the smallest field and depth of view but can scan objects from 0.5 cm to 20 cm in size with the highest accuracy (up to 40 microns) and resolution (up to 0.15 mm).

### 1) Description of Parameters

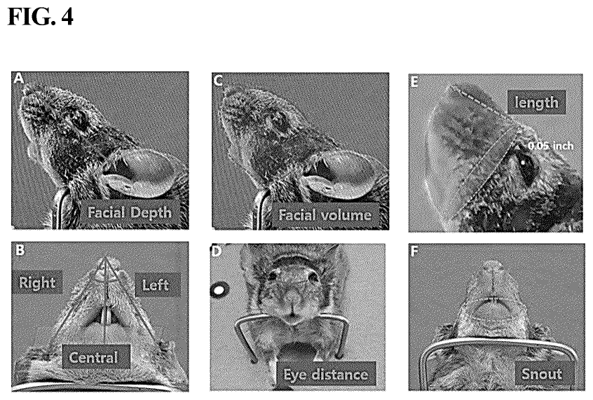

The depth drawn vertically from the nose to the projected plane including the front of ears was defined as the facial depth. The distance from the nose to the front line of the right ear was defined as the right facial distance and the left side as the left facial distance (FIGS. 4A and B). The volume from the nose to the beginning of both ears was defined as the facial volume and measured (FIG. 4C). The eye-to-eye distance was measured as the circumference of the curve along the facial surface after drawing a straight line between both eyes in the top-view (FIG. 4D). The part of the nose and mouth protruding from the face of some animals is called the snout, and the front of the eyes is the boundary. The length of the snout was measured by 3D scanning (FIGS. 4E and F). The length of snout was measured as the distance from 0.05 inches below the eye base to the tip of the nose (FIG. 4E).

### 6. Study Design

To investigate whether the facial local application of an IDS enzyme via new recombinant protein drugs can prevent or cure facial dysmorphism, different therapeutic approaches were implemented. Thus, mice were placed into two groups as follows.

The infusion interval of IDS-β for facial SC and IV injection was determined based on previous pharmacokinetic studies for preventing drug toxicity. The infusion intervals for facial SC and IV injection were bi-weekly and weekly, respectively. To prevent mortality due to the maximal dose toxicity of IDS-β, the interval between IV and SC infusions was at least 72 hours.

The “A” group of MPS II mice (aged 4 weeks: n=30; five mice per group) received HA combined with IDS-β at doses of 10, 5, 2.5, or 0.5 mg/kg bi-weekly at the mouse face injection sites for a total of two months. Groups of untreated, age-matched. MPS II (n=5), and WT (n=5) mice were used as controls.

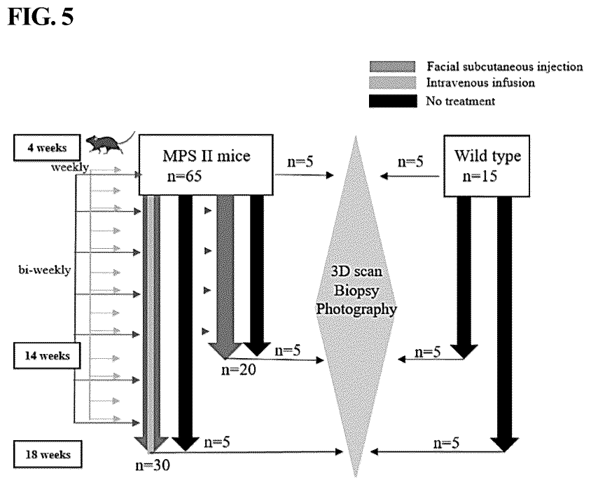

The “B” group of MPS II mice (aged 4 weeks; n=40; five mice per group) received IDS-β (0.5 mg/kg) pretreatment via the tail vein weekly 72 hours before facial SC injection for a total of three months. In addition, they received HA combined with IDS-β, a new recombinant drug, at doses of 10, 5, 2.5, or 0.5 mg/kg bi-weekly at the face injection sites. Groups of untreated, age-matched, MPS II mice (n=5), and WT (n=5) mice were used as controls. The other control groups, MPS II mice (n=5) only received IDS-β (0.5 mg/kg) facial SC injections bi-weekly and the other MPS II mice (n=5) only had IDS-β (0.5 mg/kg) IV injections weekly with bi-weekly vehicle face injections. Table 2 and FIG. 5 show the study protocol.

Two weeks after the last facial SC injection, the mice received a 3D scan with their chin against the structure on a grid plane, and the scanner rotated 360° for 2-3 minutes to take 3D pictures. Then, the animals were sacrificed via an intraperitoneal injection of Zoletil (50 mg/kg) and xylazine (10 mg/kg) and facial tissue extraction started for s-GAG analysis. Transcardial perfusion was performed with ice-cold 0.9% saline and harvested tissues were stored at −80° C. prior to biochemical analysis. For histological analysis, tissues were fixed with 4% paraformaldehyde overnight at 4° ° C.

TABLE 2

| Group | | | | | | |
|---|---|---|---|---|---|---|
| Group | | | | | | At |
| (start | | Dose (mg/kg) | | Duration | | age |
| age, wk) | Subgroup | SC + HA | IV | (months) | Assessment | (wk) |
| A (4 wk) | A(1), N = 5 | 10 + 40 | | 2 | Tissue s-GAG | 14 wk |
| | A(2), N = 5 | 5 + 40 | | | 3D scan | 14 wk |
| | A(3), N = 5 | 2.5 + 40 | | | Photography | 14 wk |
| | A(4), N = 5 | 0.5 + 40 | | | | 14 wk |
| | A(5), N = 5 | | | | | 14 wk |
| WT | A(6), N = 5 | | | | | 14 wk |
| B (4 wk) | B(1), N = 5 | 10 + 40 | 0.5 | 3 | Tissue s-GAG | 18 wk |
| | B(2), N = 5 | 5 + 40 | 0.5 | | 3D scan | 18 wk |
| | B(3), N = 5 | 2.5 + 40 | 0.5 | | Photography | 18 wk |
| | B(4), N = 5 | 0.5 + 40 | 0.5 | | | 18 wk |
| | B(5), N = 5 | Vehicle | 0.5 | | | 18 wk |
| | B(6), N = 5 | 0.5 + 40 | | | | 18 wk |
| | B(7), N = 5 | | | | | 18 wk |
| WT | B(8), N = 5 | | | | | 18 wk |

Study Protocol. wk; week(s),
WT; wild type,
SC; subcutaneous,
HA; hyaluronic acid,
IV; intravenous,
s-GAG; sulfated GAG.

### 7. Statistical Analysis

The hypothesis testing of the comparison between the groups and the corresponding descriptive statistics was performed using a non-parametric method due to the small number of mice. The primary hypothesis for the multiple pairwise comparison of each dose for the SC-treated group with the non-treated MPS II group was tested using the non-parametric Dunnett test. The other two primary hypotheses regarding the SC and IV-treated groups were also tested using the non-parametric Dunnett test and the p-value from each hypothesis test was corrected with Bonferroni's method to control the size of the type I error because these two hypotheses only differed in terms of the control group compared with the same group of each dose for the SC- and IV-treated groups. For the secondary hypotheses, multiple pairwise comparisons and other types of multiple comparisons among the different groups were performed using non-parametric Tukey's test and Mann-Whitney test with Bonferroni's correction, respectively, for continuous outcomes. Multiple comparisons among groups for categorical outcomes were performed with Fisher's exact test using the permutation method. The comparison between the two groups with no multiple comparisons was performed with the Mann-Whitney test and Fisher's exact test for continuous and categorical outcomes, respectively. Descriptive statistics are expressed as the median (minimum, maximum) for continuous variables and number (%) for categorical variables. Correlation of s-GAG or dermis thickness with facial volume was presented as a correlation coefficient and the corresponding p-value using Pearson's correlation analysis due to the normality of each analysed outcome in the SC-treated total group, and SC- and IV-treated total group. Normality was tested using the Shapiro-Wilk test. Two-sided p-values<0.05 were considered significant. SAS (Version 9.4 or higher, Enterprise BI Server, SAS Institute Inc., Cary, NC. USA) and R (Version 4.2.0) were used for all analyses.

### Results

### 1. Facial Dysmorphic Phenotype of Mice on Photography and Pathology

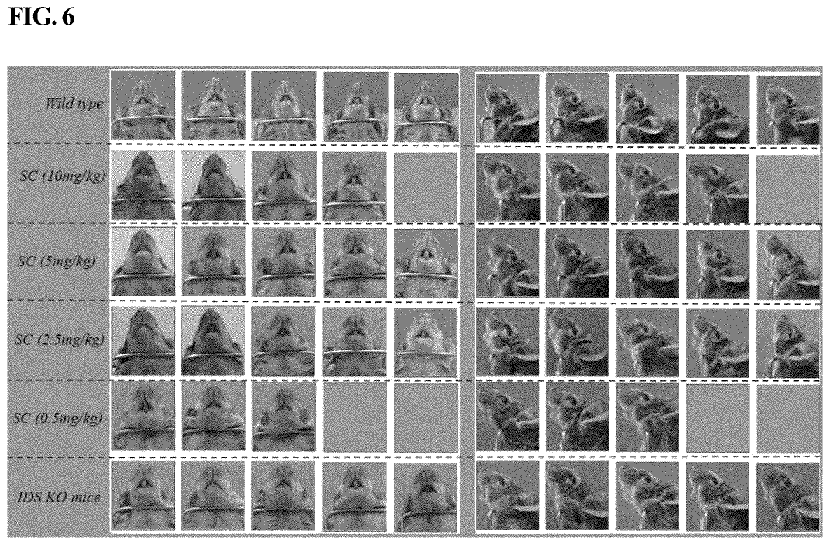

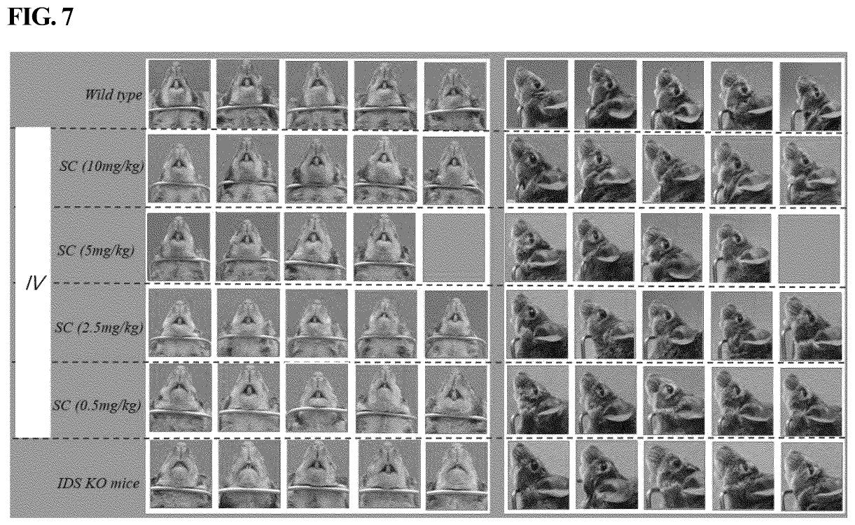

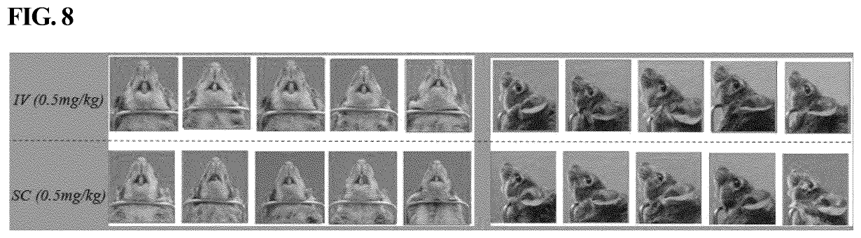

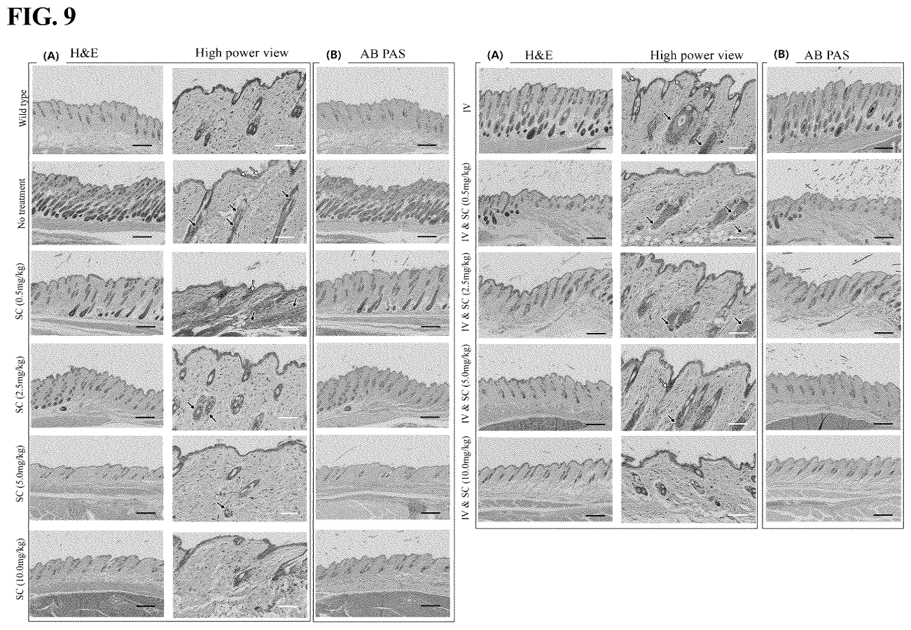

Among the study mice (n=80), MPS II mice (n=5) and WT mice (n=5) at 4 weeks of age were sacrificed for the baseline evaluation of facial dysmorphology. In addition, four mice that died were also examined in follow-up loss. Therefore, excluding these 14 mice, data from 66 mice were collected from 4 weeks to either 14 or 18 weeks of age. The median age was 29 days (range 28-31 days), and the median body weight was 14.8 g (13.4-16.2 g) (male, 100%). At 12 weeks of age, the median body weight of the mice was 26.2 g (22.6-29.4 g) and the median body weight gain (%) during the eight weeks was 75.3% (40.2-125.2%). Among the parameters measured by 3D scanning, the largest facial volume values in each of the groups A and B were A5 and B7 in the non-treated MPS II mouse group (Table 3). Similarly, A5 and B7 showed the highest s-GAG and dermis thickness as a result of pathology, and A3 and B5 among the treated MPS II mouse groups. The snout-angle view in FIG. 6 shows that the higher the facial SC dose, the sharper the snout angle as in WT mice. FIG. 7 shows that the overall snout angle was sharper than that shown in FIG. 6 and evidently the higher facial SC dose, the more similar the angle was to WT mice. In FIG. 8, the snout angle tended to be sharper in the SC group than in the IV-treated group. The non-treated MPS II mice had more anagenic hair follicles, skin wrinkles, and increased skin thickness (FIG. 9). In the case of GAG accumulation analysed by AB PAS, the staining intensity was increased in the epidermal keratinocytes and anagenic hair follicles in non-treated MPS II mice (FIG. 9B). The IV-treated group seemed to be similar to the non-treated MPS II mice in terms of skin architecture (FIG. 9A). However, the facial SC-treated group showed a similar architecture to normal skin and the wrinkles and thickness of the skin layers were also reduced. In the IV-and-SC-treated MPS II mice, skin wrinkles and thickness were similar to those of WT mice (FIG. 9A), as was the signal intensity of AB PAS staining (FIG. 9B).

TABLE 3

| | | | | | | | | | | | Median |
|---|---|---|---|---|---|---|---|---|---|---|---|
| | | Median (Min-Max) | | | | | | | | | (Min-Max) |
| | | Facial | Facial | Eye | Snout | Snout | Sulfated | Dermis | | | Weight |
| | | volume | depth | distance | cir. | length | GAG | thickness | N (%) | | Gain |
| Group | N | (mm³) | (mm) | (mm) | (mm) | (mm) | (μg/μL) | (μm) | Alive/Death | | (%)* |
| A1 | 4 | 1644.61 | 18.62 | 14.85 | 39.58 | 9.55 | 0.37 | 996 | 4 | 1 | 54.47 |
| | | (1458.06- | (18.19- | (14.62- | (38.27- | (9.39- | (0.32- | (965- | (80) | (20) | (40.24- |
| | | 1664.31) | 19.04) | 15.27) | 39.83) | 9.91) | 0.48) | 1102) | | | 63.58) |
| A2 | 5 | 1685.03 | 18.44 | 13.6 | 38.19 | 9.28 | 0.34 | 1103 | 5 | 0 | 73.97 |
| | | (1628.78- | (17.59- | (12.7- | (37.63- | (8.93- | (0.26- | (998- | (100) | (0) | (56.94- |
| | | 1817.11) | 19.22) | 14.89) | 39.53) | 9.5) | 0.44) | 1222) | | | 81.56) |
| A3 | 5 | 1879.58 | 18.2 | 14.82 | 39.82 | 9.41 | 0.44 | 1248 | 5 | 0 | 60.93 |
| | | (1700.33- | (17.62- | (13.09- | (39.66- | (8.93- | (0.26- | (1125- | (100) | (0) | (46.71- |
| | | 1911.1) | 19.37) | 15.79) | 39.89) | 9.86) | 0.55) | 1321) | | | 76.77) |
| A4 | 3 | 1917.99 | 18.14 | 14.69 | 41.05 | 9.61 | 0.42 | 1203 | 3 | 2 | 59.62 |
| | | (1746.67- | (16.91- | (14.5- | (39.13- | (9.39- | (0.42- | (1198- | (60) | (40) | (58.49- |
| | | 2066.62) | 18.33) | 15.11) | 41.83) | 9.62) | 0.59) | 1253) | | | 62.41) |
| A5 | 5 | 2243.97 | 17.38 | 15.34 | 42.12 | 9.56 | 0.51 | 1256 | 5 | 0 | 73.78 |
| | | (2007.64- | (16.31- | (14.68- | (41.28- | (8.93- | (0.44- | (1221- | (100) | (0) | (63.58- |
| | | 2491.71) | 18.39) | 16.42) | 42.39) | 10.38) | 0.58) | 1369) | | | 90.41) |
| A6 | 5 | 1495.69 | 19.47 | 14.5 | 38.99 | 9.66 | 0.28 | 936 | 5 | 0 | 73.65 |
| | | (1301.74- | (18.09- | (14.07- | (37.56- | (9.08- | (0.21- | (854- | (100) | (0) | (52.17- |
| | | 1588.33) | 20.17) | 15.49) | 39.39) | 9.93) | 0.31) | 998) | | | 80.82) |
| B1 | 5 | 1515.61 | 18.84 | 14.43 | 40.02 | 9.57 | 0.37 | 985 | 5 | 0 | 88.36 |
| | | (1409.15- | (17.29- | (12.59- | (38.98- | (9.44- | (0.26- | (877- | (100) | (0) | (47.31- |
| | | 1576.81) | 20.12) | 15.31) | 40.3) | 9.91) | 0.41) | 1023) | | | 114.6) |
| B2 | 4 | 1583.44 | 18.41 | 14.66 | 40.05 | 9.67 | 0.29 | 989 | 4 | 1 | 92.19 |
| | | (1539.89- | (17.85- | (14.07- | (38.86- | (9.46- | (0.26- | (978- | (80) | (20) | (62.94- |
| | | 1621.89) | 19.18) | 15.53) | 42.35) | 10.06) | 0.38) | 1102) | | | 103.52) |
| B3 | 5 | 1682.4 | 17.83 | 14.16 | 40.22 | 9.64 | 0.35 | 1025 | 5 | 0 | 104.23 |
| | | (1554.79- | (17.25- | (13.86- | (37.65- | (9.44- | (0.23- | (956- | (100) | (0) | (53.3- |
| | | 1723.84) | 19.21) | 15.65) | 41.51) | 10.2) | 0.44) | 1174) | | | 112.59) |
| B4 | 5 | 1684.94 | 17.82 | 14.44 | 39.13 | 9.7 | 0.4 | 1166 | 5 | 0 | 90.77 |
| | | (1631.15- | (16.98- | (12.56- | (37.4- | (9.05- | (0.21- | (1085- | (100) | (0) | (57.23- |
| | | 1795.14) | 18.75) | 15.3) | 39.76) | 10.07) | 0.5) | 1255) | | | 107.69) |
| B5 | 5 | 1779.21 | 17.62 | 15.05 | 39.74 | 9.63 | 0.49 | 1235 | 5 | 0 | 76.51 |
| | | (1742.9- | (17.42- | (13.53- | (37.51- | (9.33- | (0.41- | (1223- | (100) | (0) | (55.92- |
| | | 1844.63) | 18.46) | 15.27) | 41.75) | 9.93) | 0.5) | 1255) | | | 83.33) |
| B6 | 5 | 1677.2 | 17.76 | 14.42 | 40.33 | 9.53 | 0.43 | 1102 | 5 | 0 | 65.54 |
| | | (1608.88- | (17.11- | (13.89- | (39.09- | (9.29- | (0.36- | (1023- | (100) | (0) | (62.03- |
| | | 1702.56) | 18.04) | 15.63) | 41.21) | 10.01) | 0.44) | 1191) | | | 76.98) |
| B7 | 5 | 2144.45 | 17.3 | 14.88 | 40.87 | 9.54 | 0.51 | 1340 | 5 | 0 | 96.5 |
| | | (1948.23- | (16.26- | (14.26- | (37.83- | (8.16- | (0.48- | (1223- | (100) | (0) | (72.08- |
| | | 2327.53) | 18.19) | 16.16) | 41.29) | 10.04) | 0.56) | 1405) | | | 107.14) |
| B8 | 5 | 1437.51 | 19.06 | 14.08 | 37.49 | 9.05 | 0.3 | 918 | 5 | 0 | 100 |
| | | (1257.59- | (18.61- | (12.74- | (37- | (8.7- | (0.25- | (803- | (100) | (0) | (76.28- |
| | | 1481.69) | 19.66) | 14.44) | 38.45) | 9.46) | 0.34) | 966) | | | 125.2) |
| A5 + B7 | 10 | — | — | — | — | — | — | — | 10 | 0 | 81.22 |
| | | | | | | | | | (100) | (0) | (63.58- |
| | | | | | | | | | | | 107.14) |
| A1 + A2 + | 56 | — | — | — | — | — | — | — | 56 | 4 | 75.13 |
| A3 + A4 + | | | | | | | | | (93.33) | (6.67) | (40.24- |
| B1 + B2 + | | | | | | | | | | | 125.20) |
| B3 + B4 + | | | | | | | | | | | |
| B5 + B6 | | | | | | | | | | | |
| A1 + A2 + | | — | — | — | — | — | — | — | 17 | 3 | 60.93 |
| A3 + A4 | | | | | | | | | (85) | (15) | (40.24- |
| | | | | | | | | | | | 81.56) |
| B1 + B2 + | | — | — | — | — | — | — | — | 19 | 1 | 91.67 |
| B3 + B4 | | | | | | | | | (95) | (5) | (47.31- |
| | | | | | | | | | | | 114.60) |
| N_WT | 5 | 1054.45 | 16.75 | 13.35 | 36.37 | 8.4 | 0.4 | 884 | — | — | — |
| | | (883.49- | (15.58- | (11.82- | (34.53- | (7.69- | (0.27- | (855.33- | | | |
| | | 1214.9) | 18.26) | 13.87) | 36.9) | 9.09) | 0.46) | 935.67) | | | |
| N_KO | 5 | 1371.34 | 15.98 | 14.04 | 36.96 | 8.86 | 0.51 | 1122 | — | — | — |
| | | (1205.81- | (14.98- | (13.94- | (34.81- | (8.22- | (0.46- | (947.67- | | | |
| | | 1604.16) | 16.99) | 14.26) | 39.23) | 9.56) | 0.61) | 1148.67) | | | |

Demographic table of the A and B mouse groups in this study. Min: minimum, Max: maximum, Snout cir.: Snout circumference, N_WT: wild-type mice at 4 weeks of age, N_KO; MPS II mice at 4 weeks of age, *Weight gain (%) from 4 weeks to 12 weeks of age.

### 2. Morphology Comparison Between MPS II Mice and WT Mice

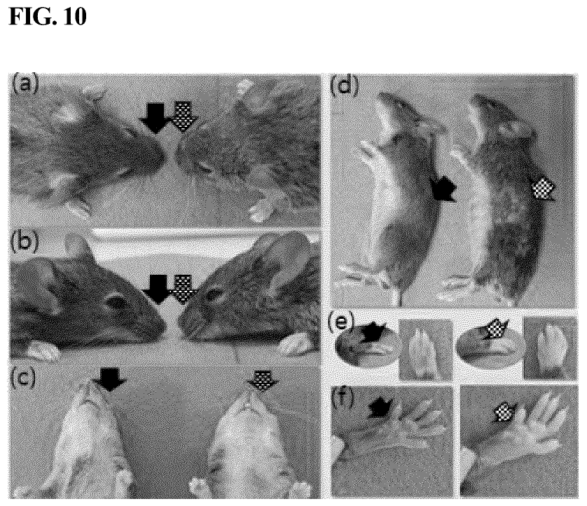

The MPS II mice had coarse facial features and their tongues were enlarged. At necropsy, the MPS II mice had a biopsy of the facial subcutaneous tissue. Light microscopy disclosed abundant intracytoplasmic clear vacuoles and marked cytoplasmic enlargement, with the accumulation of pale, fine granular material. This storage material was identified as dermis mucopolysaccharide by AB PAS stains (FIG. 9). These cells contained PAS-positive storage material. FIG. 10 shows a photograph of an MPS II mouse weighing 30.67 g and a WT mouse weighing 30.70 g at the same age. MPS II mice had shorter facial depths, and a larger snout angle and circumference than the WT mice (FIG. 10a, b, and c). Furthermore, they had coarse hair over their whole bodies and partial alopecia (FIG. 10d). The fore and hind paws were more rounded in shape and curled under than the WT mice (FIGS. 10e and f).

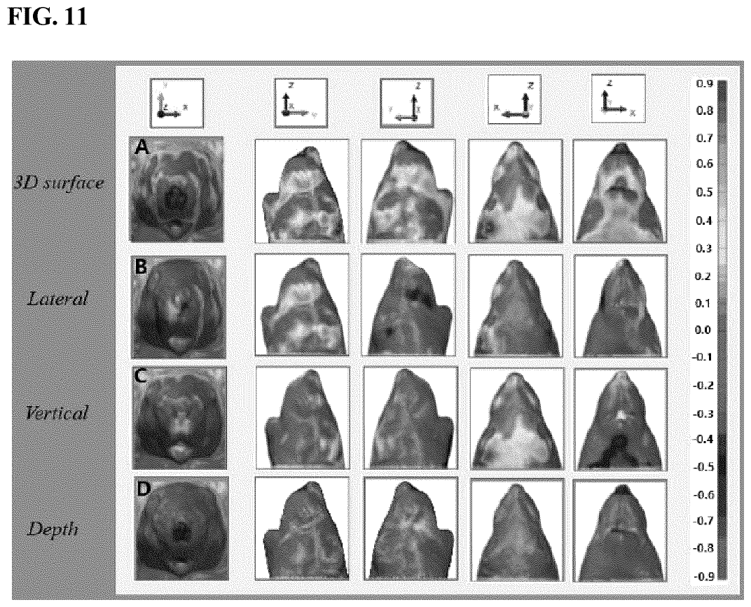

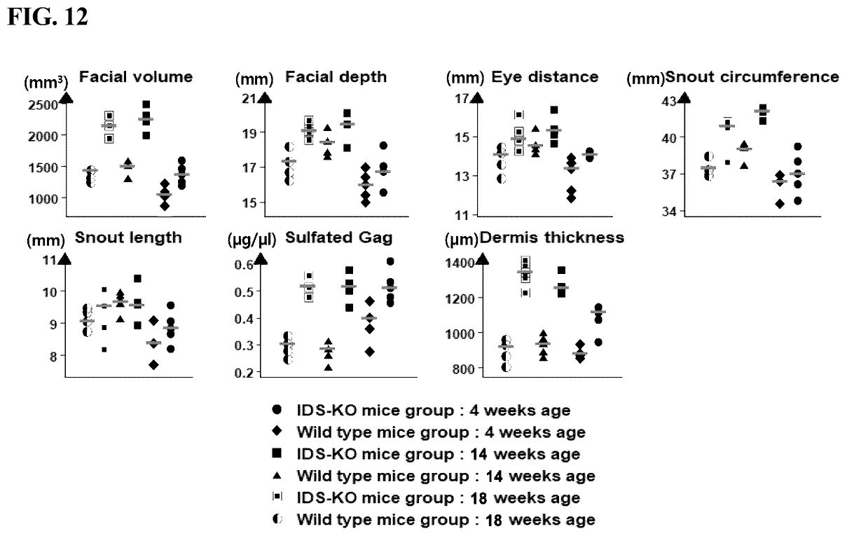

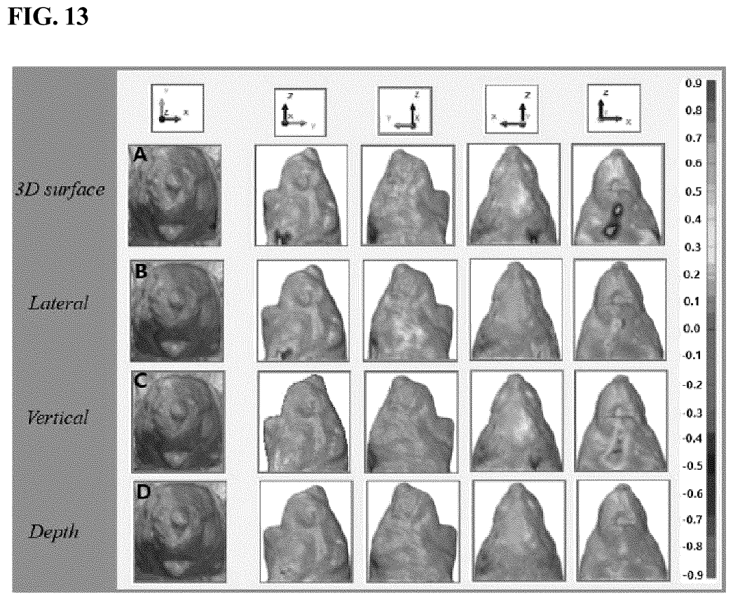

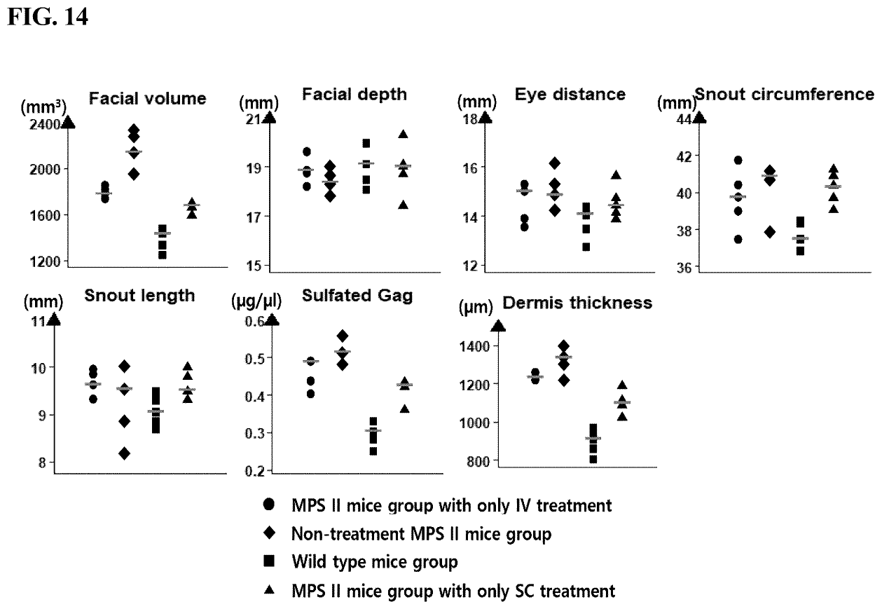

3D imaging (FIG. 11) showed MPS II mice had shorter depths between the two groups in the depth direction, so the tip of the nose was in blue. However, in the lateral and vertical directions, MPS II mice had a larger value, which is shown in red. Therefore, the facial depths of MPS II mice were shorter than those of WT mice, but the overall face volume was larger on the lateral and vertical sides. The graphs in FIG. 12 show the dysmorphic facial parameter values for MPS II and WT groups at 4, 14, and 18 weeks of age, respectively. There were significant differences in the parameters of facial volume, s-GAG, and dermis thickness between the groups (Table 4).

TABLE 4

| | | B7 | A5 | N_WT |
|---|---|---|---|---|
| | | vs. B8 | vs. A6 | vs. N_KO |
| Age (weeks) | | 18 | 14 | 4 |
| p-value | Facial volume | 0.008* | 0.008* | 0.016* |
| | Facial depth | 0.008* | 0.056 | 0.310 |
| | Eye distance | 0.016* | 0.222 | 0.008* |
| | Snout circumference | 0.032* | 0.008* | 0.222 |
| | Snout length | 0.403 | 0.834 | 0.531 |
| | Sulfated GAG | 0.008* | 0.008* | 0.016* |
| | Dermis thickness | 0.008* | 0.008* | 0.008* |

Statistical analysis of the dysmorphic facial parameter values for MPS II and WT groups at 4, 14, and 18 weeks of age, respectively. P-values were calculated by the Mann-Whitney test. B7; MPS II mice at 18 weeks of age, B8; WT mice at 18 weeks of age, A5; MPS II mice at 14 weeks of age, A6; WT mice at 14 weeks of age, N_WT; WT mice at 4 weeks of age, N_KO; MPS II mice at 4 weeks of age, *p-value<0.05

3. The difference between treated and non-treated groups
3-1. IV-Treated Vs. Non-Treated MPS II Mouse Groups

The facial dysmorphology of non-treated IDS KO mice (median weight: 26.5 g (25.4-28.1 g)) in FIG. 7 and IV-treated MPS II mice (median weight: 25.3 g (23.7-27.3 g)) showed a similar phenotype. The facial depth and lateral and vertical values changed in the ranges of 0.0-0.5, ⁻0.4⁺0.7, and ⁻0.4-⁺0.7, respectively. Overall, there was no significant difference in the facial dysmorphology between the two groups as the green area (0.0-+0.2) occupied most of them.

The median differences in facial volume (−365.2 mm³), depth (+0.65 mm), snout circumference (−1.13 mm), s-GAG (−0.03 μg/μl), and dermis thickness (−105 μm) in the IV-treated group were lower than in the non-treated MPS II group. However, Table 5 shows no statistically significant differences.

TABLE 5

| | | B5 vs. B7† |
|---|---|---|
| Age (weeks) | | 18 |
| p-value | Facial volume | 0.071 |
| | Facial depth | 0.944 |
| | Eye distance | >0.999 |
| | Snout circumference | >0.999 |
| | Snout length | >0.999 |
| | Sulfated GAG | 0.944 |
| | Dermis thickness | >0.999 |

Statistical analysis of the dysmorphic facial parameter values for MPS II mice with intravenous treatment (B5) and the non-treated group (B7). † P-values were calculated using the Mann-Whitney test with Bonferroni's correction.

3-2. SC-Treated Vs. Non-Treated MPS II Mouse Groups

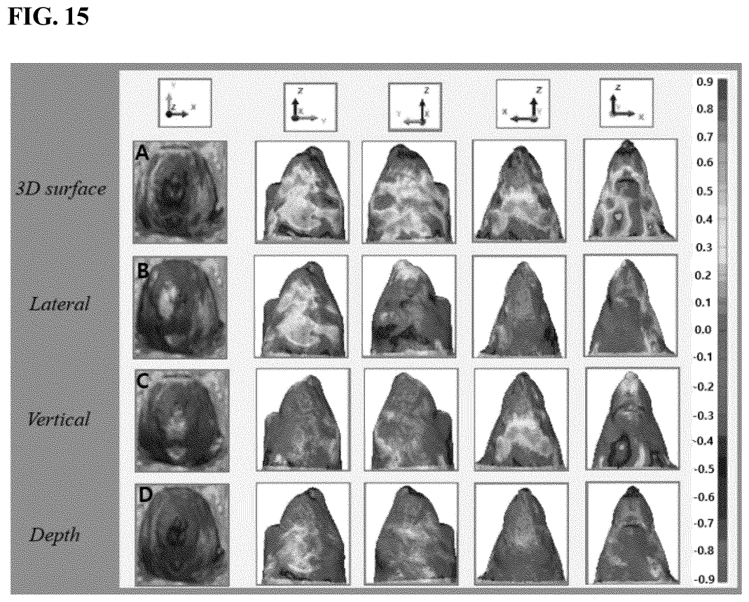

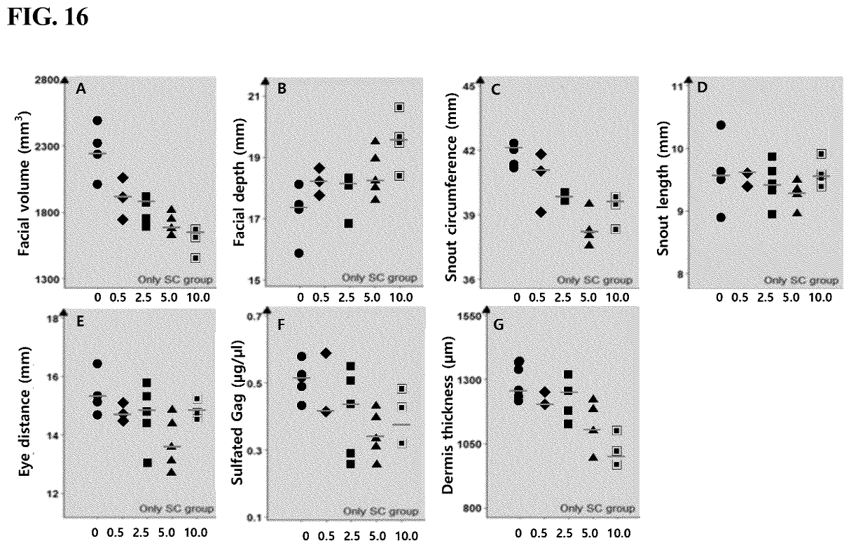

Among the facial morphology of the non-treated (median weight 28.3 g (24.4-28.6 g)) and SC-treated MPS II groups (median weight: 25.2 g (22.6-28.5 g)) (FIGS. 7 and 8), the SC-treated group showed a sharper snout angle than the non-treated group. In FIG. 15, the changes of 3D surface, facial depth, and lateral and vertical directions were in the ranges ⁻0.8-⁺0.8, ⁻0.7-⁺0.4, ⁻0.5-⁺0.6, and ⁻0.8-⁺0.7, respectively. Around the snout showed a negative value, whereas other areas of the face showed positive values.

The median differences in facial volume (−467.3 mm³), snout circumference (−0.54 mm), snout length (−0.01 mm), eye distance (−0.46 mm), s-GAG (−0.03 μg/μl), and dermis thickness (−105 μm) in the SC-treated group were lower than in the non-treated MPS II group.

In Table 6, the non-treated MPS II group showed a statistically significant difference in facial volume with the 10, 5, and 2.5 mg/kg groups of the facial SC injection dose but not with the 0.5 mg/kg group (p=0.1). There was a statistically significant difference in the dermis thickness results compared to A1, which was the highest dose of 10 mg/kg (p=0.022) and in snout circumference when compared with A2 (p=0.024).

In Table 6-1 comparing facial SC injection dose subgroups, there were significant differences in facial volume between A1 and A3 (p=0.044) and A1 and A4 (p=0.049). In addition, there were significant differences in dermis thickness between A1 and A3 (p=0.008), A1 and A4 (p=0.004), and A2 and A3 (p=0.001).

TABLE 6

| | | A5 | A5 | A5 | A5 |
|---|---|---|---|---|---|
| | | vs. A1 | vs. A2 | vs. A3 | vs. A4 |
| Age (weeks) | | 14 | | | |
| p-value | Facial volume† | 0.001* | 0.005* | 0.004* | 0.101 |
| | Facial depth‡ | 0.191 | 0.520 | 0.268 | 0.799 |
| | Eye distance‡ | 0.665 | 0.103 | 0.770 | 0.534 |
| | Snout | 0.051 | 0.024* | 0.082 | 0.460 |
| | Circumference‡ | | | | |
| | Snout length‡ | >0.999 | 0.316 | 0.978 | >0.999 |
| | Sulfated GAG ‡ | 0.231 | 0.134 | 0.518 | 0.887 |
| | Dermis thickness‡ | 0.022* | 0.996 | 0.718 | 0.353 |

Statistical analysis of the dysmorphic facial parameter values for MPS II mice in the facial subcutaneous (SC) injection and non-treated MPS II groups (A5). A1; facial SC dose, 10 mg/kg, A2; facial SC dose, 5.0 mg/kg, A3; facial SC dose, 2.5 mg/kg, and A4; facial SC dose, 0.5 mg/kg. † P-values were calculated using the non-parametric Dunnett test. ‡ P-values were calculated using the non-parametric Tukey test, *p-value<0.05.

TABLE 6-1

| | | Al vs. | | | A2 vs. | | |
|---|---|---|---|---|---|---|---|
| | | A2 | A3 | A4 | A3 | A4 | A3 VS. A4 |
| Age (weeks) | | 14 | | | | | |
| p- | Facial volume | 0.610 | 0.044* | 0.049* | 0.344 | 0.251 | 0.729 |
| value | Facial depth | 0.626 | 0.926 | 0.322 | 0.898 | 0.928 | 0.394 |
| | Eye distance | 0.258 | 0.100 | 0.984 | 0.557 | 0.504 | 0.999 |
| | Snout | 0.224 | 0.412 | 0.757 | 0.060 | 0.249 | 0.996 |
| | circumference | | | | | | |
| | Snout length | 0.121 | 0.971 | >0.999 | 0.555 | 0.166 | 0.977 |
| | Sulfated GAG | 0.876 | 0.993 | 0.873 | 0.819 | 0.559 | 0.977 |
| | Dermis thickness | 0.135 | 0.008* | 0.004* | 0.001* | 0.309 | 0.272 |

Statistical analysis of the dysmorphic facial parameter values for each group of 0.5 (A4), 2.5 (A3), 5.0 (A2), and 10 mg/kg (A1) according to the facial subcutaneous injection to MPS II mice. P-values were calculated using the non-parametric Tukey test, *p-value<0.05.

3-3. IV-and-SC-Treated Vs. Non-Treated MPS II Groups

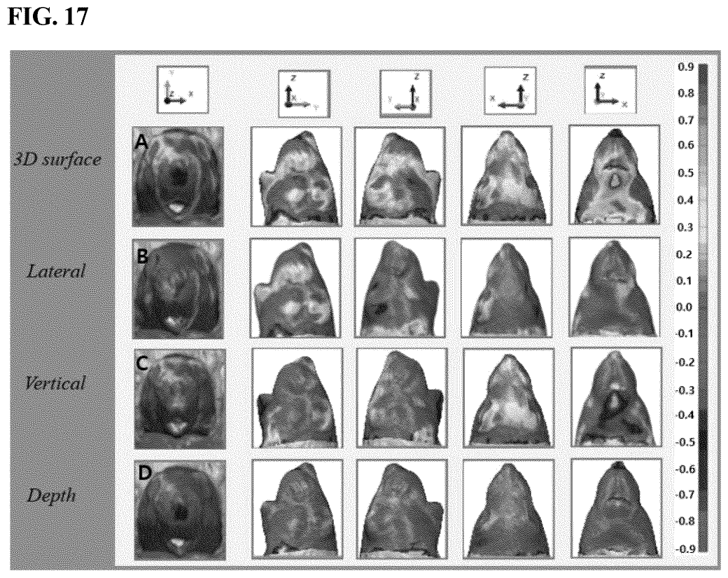

Regarding the facial morphology of the non-treated (median weight 26.5 g (25.4-28.1 g)) and IV-and-SC-treated MPS II mice (median weight 26.4 g (24.3-29.4 g)) (FIG. 7), the IV-and-SC-treated group showed a sharper snout angle than the non-treated group. As shown in FIG. 17, changes in the 3D surface, facial depth, and lateral and vertical direction were in the range of −0.9 to 0.9, −0.5 to 0.3, −0.8 to 0.7, and −0.8 to 0.8, respectively. The area around the snout showed a negative value, whereas other areas of the face showed positive values.

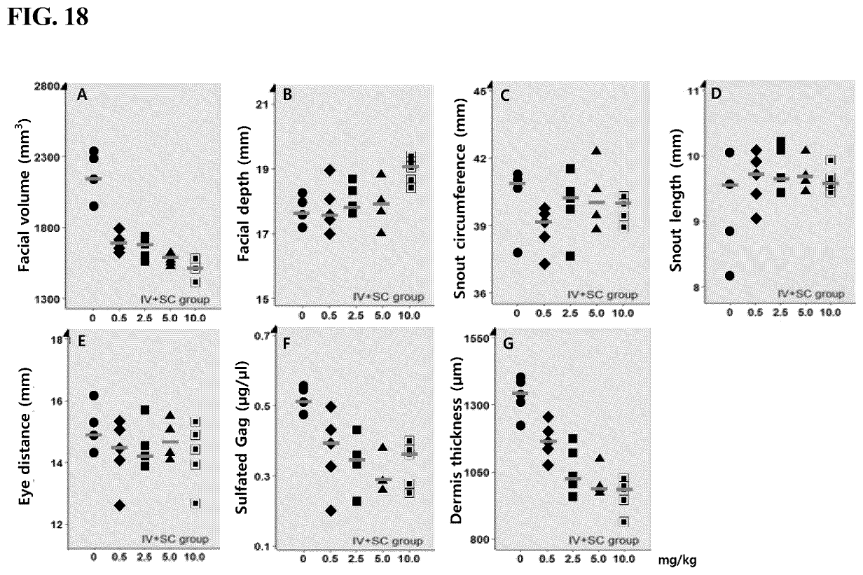

The median differences in facial volume (−532.3 mm³), snout circumference (−1.1 mm), eye distance (−0.54 mm), s-GAG (−0.17 μg/μl), and dermis thickness (−327.5 μm) in the IV-and-SC-treated group were lower than those in the non-treated MPS II mouse group (FIG. 18, Table 3).

As shown in Table 7, there were statistically significant differences in facial volume between the B1, B2, B3, and B4 groups of the IV-and-SC-treated group and the non-treated MPS II mouse group (p<0.05). Table 7-1 comparing facial SC injection dose subgroups showed no significant differences in facial dysmorphism parameters.

TABLE 7

| | | B7 | B7 | B7 | B7 |
|---|---|---|---|---|---|
| | | vs. B1 | vs. B2 | vs. B3 | vs. B4 |
| Age (weeks) | | 18 | | | |
| p-value | Facial volume | <0.001* | <0.001* | <0.001* | <0.001* |
| | Facial depth | 0.134 | 0.540 | 0.135 | 0.944 |
| | Eye distance | >0.999 | >0.999 | >0.999 | >0.999 |
| | Snout circumference | >0.999 | >0.999 | >0.999 | >0.999 |
| | Snout length | >0.999 | >0.999 | >0.999 | >0.999 |
| | Sulfated GAG | 0.135 | 0.270 | 0.135 | 0.270 |
| | Dermis thickness | 0.207 | 0.340 | 0.207 | 0.367 |

Statistical analysis of dysmorphic facial parameter values for MPS II mice treated with SC and IV (0.5 mg/kg) vs. non-treated MPS II mice (B7). B1; facial SC dose, 10 mg/kg, B2; facial SC dose, 5.0 mg/kg, B3; facial SC dose, 2.5 mg/kg, and B4; facial SC dose, 0.5 mg/kg. P-values were calculated using the non-parametric Dunnett test with Bonferroni's correction, *p-value<05.

TABLE 7-1

| | | B1 vs. | | | B2 vs. | | |
|---|---|---|---|---|---|---|---|
| | | B2 | B3 | B4 | B3 | B4 | B3 vs. B4 |
| Age (weeks) | | 18 | | | | | |
| p- | Facial volume | 0.571 | 0.143 | 0.071 | >.999 | 0.143 | >.999 |
| value | Facial depth | >.999 | >.999 | >.999 | >.999 | >.999 | >.999 |
| | Eye distance | >.999 | >.999 | >.999 | >.999 | >.999 | >.999 |
| | Snout | >.999 | >.999 | >.999 | >.999 | >.999 | >.999 |
| | circumference | | | | | | |
| | Snout length | >.999 | >.999 | >.999 | >.999 | >.999 | >.999 |
| | Sulfated GAG | >.999 | >.999 | >.999 | >.999 | >.999 | >.999 |
| | Dermis thickness | >.999 | >.999 | 0.207 | >.999 | 0.635 | >.999 |

Statistical analysis of dysmorphic facial parameter values for each group of 0.5 (B4), 2.5 (B3), 5.0 (B2), and 10 mg/kg (B1) according to the facial SC injection dose in the IV-and-SC-treated MPS II group. P-values were calculated using the Mann-Whitney test with Bonferroni's correction.

### 4. Comparison Between Treated Groups

4-1. IV-Treated Vs. IV-and-SC-Treated MPS II Groups

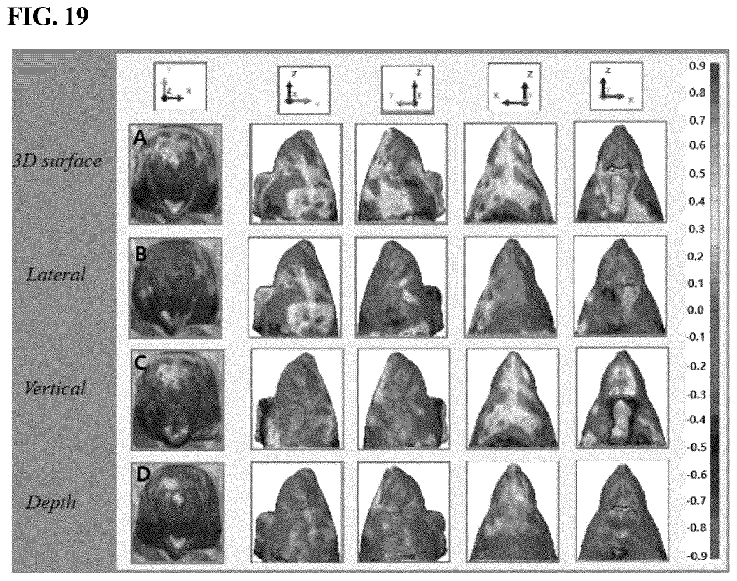

Regarding the facial morphology of IV-treated (median weight 25.3 g (23.7-27.3 g)) and IV-and-SC-treated MPS II groups (median weight 26.4 g (24.3-29.4 g)) (FIG. 7, FIG. 8), the IV-and-SC-treated group showed a sharper snout angle than the IV-treated group. As shown in FIG. 19, the changes in 3D surface, facial depth, and lateral and vertical direction were in the range of −0.8 to 0.8, −0.7 to 0.7, −0.6 to 0.8, and −0.7 to 0.7, respectively. Partially positive values were observed for the face, especially around the snout and the forehead.

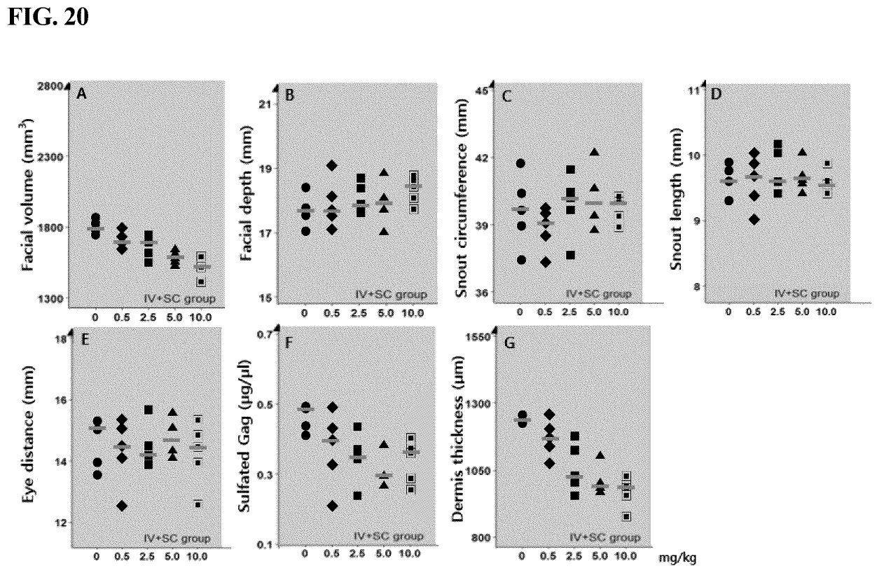

The median differences in facial volume (−167.0 mm³), snout circumference (−0.71 mm), s-GAG (−0.14 μg/μl), and dermis thickness (−222.5 μm) in the IV-and-SC-treated group were lower than those in the IV-treated MPS II group (FIG. 20, Table 3).

As shown in Table 8, there were statistically significant differences in facial volume between the B1, B2, and B3 groups of the IV-and-SC-treated group, except for B4, and the IV-treated MPS II group (p<05). The comparison with the SC-treated group (B6) showed no significant differences, whereas the comparison with the WT group (B8) showed significant differences except for the eye distance results.

TABLE 8

| | | B5 vs. | | | | | |
|---|---|---|---|---|---|---|---|
| | | B1† | B2† | B3† | B4† | B6‡ | B8 [symbol] |
| Age (weeks) | | 18 | | | | | |
| p- | Facial volume | <0.001* | <0.001* | 0.003* | 0.118 | 0.071 | 0.008* |
| value | Facial depth | 0.540 | >0.999 | >0.999 | >0.999 | >0.999 | 0.032* |
| | Eye distance | >0.999 | >0.999 | >0.999 | >0.999 | >0.999 | 0.222 |
| | Snout | >0.999 | >0.999 | >0.999 | >0.999 | >0.999 | 0.032* |
| | circumference | | | | | | |
| | Snout length | >0.999 | >0.999 | >0.999 | >0.999 | >0.999 | 0.028* |
| | Sulfated GAG | 0.135 | 0.270 | 0.540 | >0.999 | >0.999 | 0.008* |
| | Dermis | 0.207 | 0.340 | 0.207 | >0.999 | 0.207 | 0.008* |
| | thickness | | | | | | |

Statistical analysis of dysmorphic facial parameter values for IV-treated MPS II mice vs. IV-and-SC-treated (B1, B2, B3, B4), SC-treated (B6), and WT mice (B8). † P-values were calculated using the non-parametric Dunnett test with Bonferroni's correction. ‡ P-values were calculated using the Mann-Whitney test with Bonferroni's correction. [symbol] P-values were calculated using the Mann-Whitney test, *p-value<05.

4-2. SC-Treated Vs. IV-and-SC-Treated MPS II Mouse Groups

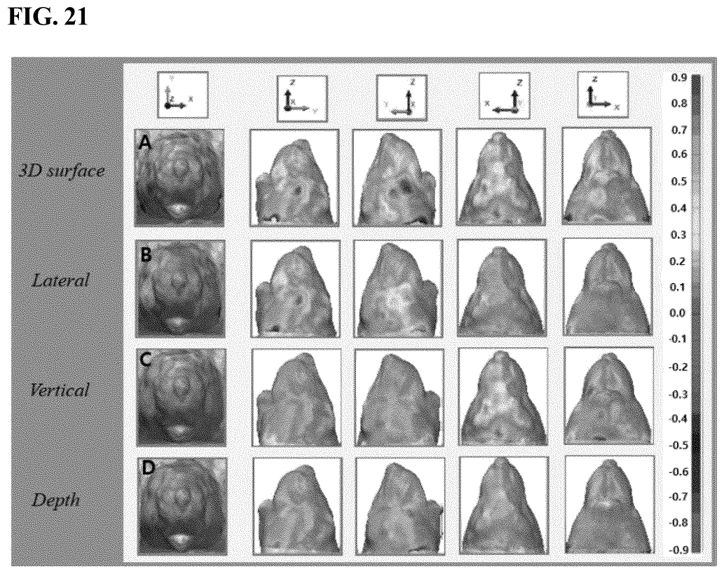

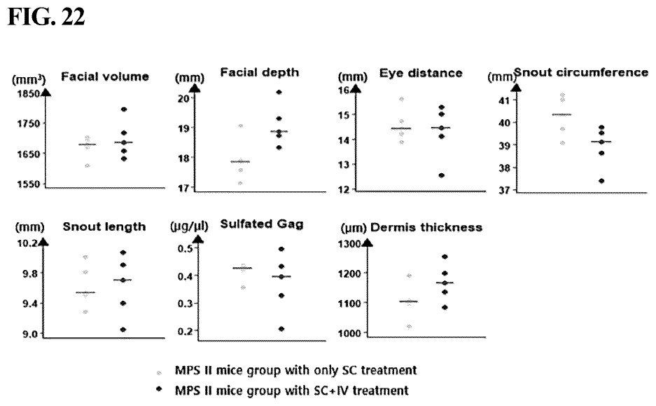

The facial dysmorphology of the IV-and-SC-treated MPS II mouse group (B4, median weight 24.8 g (24.3-26.8 g) with an SC injection dose of 0.5 mg/kg and the SC-treated mice group (B6, median weight 25.6 g (24.5-26.8 g)) seemed to be similar (FIGS. 7, 8). As shown in FIG. 21, the differences in the facial depth, lateral direction, and vertical direction were −0.5 to 0.3, −0.5 to 0.7, and −0.7 to 0.8, respectively. Moreover, the difference between the two groups was −0.7 to 0.8 in the 3D surface view, and the green colour occupied a high proportion of the facial area. FIG. 21 shows that the median values were lower in the IV-and-SC-treated group (B4) than in the group with SC injection alone (B6) in terms of snout circumference (−1.20 mm), and s-GAG (−0.03 μg/μl). The facial volume (+7.74 mm³), eye distance (+0.02 mm), snout length (+0.17 mm), and dermis thickness (+64.0 μm) had higher median values in the IV-and-SC-treated group. No statistically significant difference was found in the analysis of facial dysmorphism between the two groups (Table 9).

TABLE 9

| | | B6 vs. B4 |
|---|---|---|
| Age (weeks) | | 18 |
| p-value | Facial volume | >0.999 |
| | Facial depth | >0.999 |
| | Eye distance | >0.999 |
| | Snout circumference | >0.999 |
| | Snout length | >0.999 |
| | Sulfated GAG | >0.999 |
| | Dermis thickness | >0.999 |

Statistical analysis of the dysmorphic facial parameter values for SC-treated MPS II (B6) vs. IV-and-SC-treated (B4) mice. P-values were calculated using the Mann-Whitney test with Bonferroni's correction.

5. Monitoring of Adverse Effects in Groups that Underwent the SC Procedure

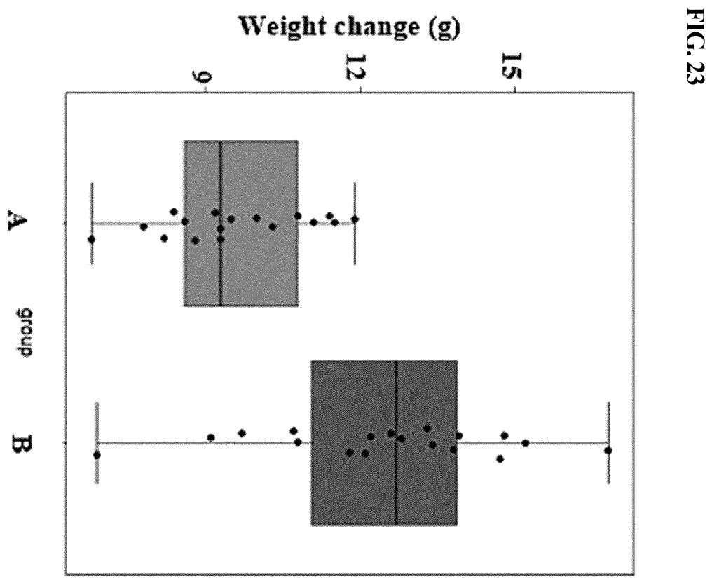

To monitor side effects of the SC procedure, mouse behaviour, appearance, dietary reduction, weight loss, and mortality were closely observed daily. The mouse behaviour was observed immediately after the SC procedure because the procedure was performed without anaesthesia. The median weight change of mice in group A that underwent the SC injection procedure was 9.3 g (6.8-11.9 g) from 4 to 12 weeks of age, and the median weight change of mice in group B was 12.6 g (8.8-15.7 g) (FIG. 23). There was a statistically significant weight gain in group B (p<0.001).

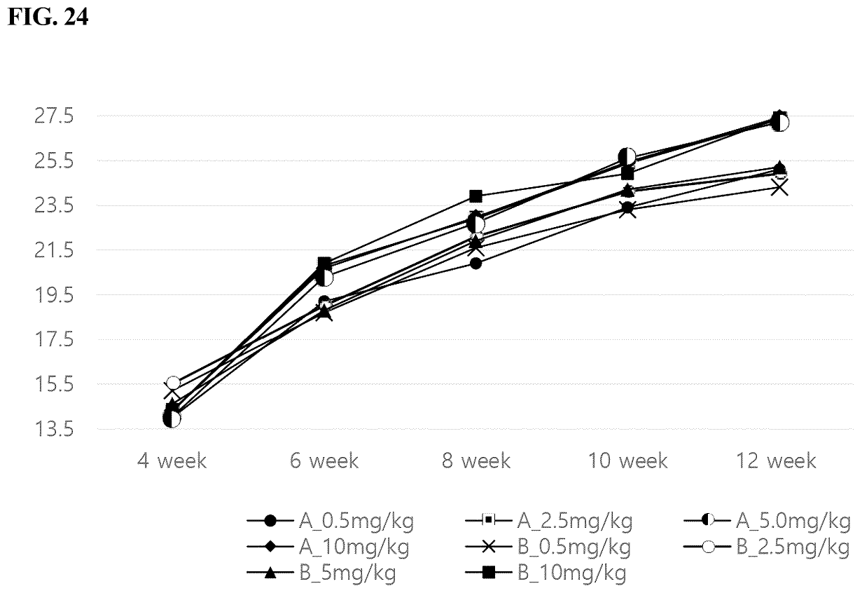

The median body weight gain values of subgroups by SC injection dose of groups A and B (FIG. 24) were 69.8% (57.2-107.7%) in the 0.5-mg/kg group (A4, B4), 69.6% (46.7-112.6%) in the 2.5-mg/kg group (A3, B3), 77.1% (56.9-103.5%) in the 5.0-mg/kg group (A2, B2), and 63.6% (40.2-114.6%) in the 10-mg/kg group (A1, B1) from 4 to 12 weeks of age. There was no significant difference in weight change (%) between groups by dose (p=0.906).

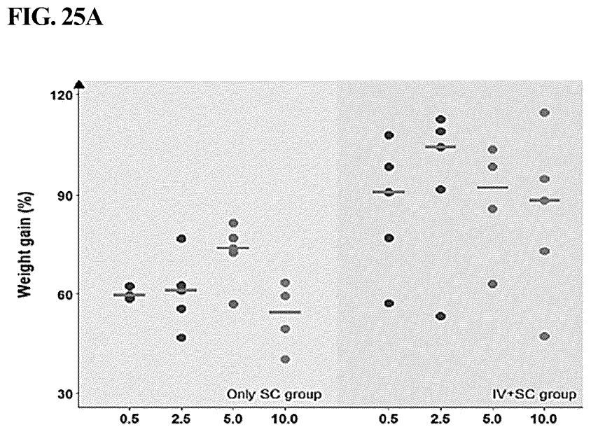

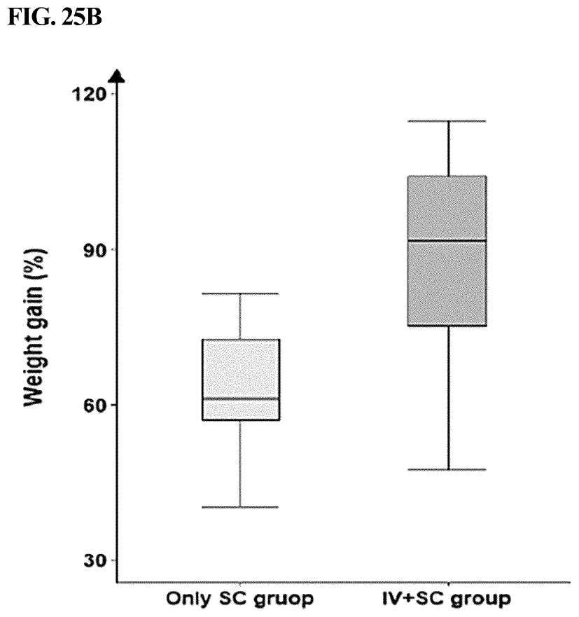

The groups that underwent the SC procedure were divided into groups that only received the SC procedure (A1, A2, A3, A4) and groups that also received the IV treatment (B1, B2, B3, B4). The median weight change (%) of the groups that only received the SC procedure was 60.93% (40.24-81.56%), and that of the groups that also received IV treatment and only IV treatment were 91.67% (47.31-114.60%) and 76.5% (55.9-83.3%), respectively (FIG. 25, Table 3). There was a significant difference between the only SC-treated and IV-and-SC-treated groups (p=0.001). However, for the comparison of weight gain (%) according to subgroups, there was a significant difference between A4 at 59.62% (58.49-62.41%) and A5 at 73.78% (63.58-90.41%) (p=0.040) (Table 10).

TABLE 10

| | A1 | A2 | A3 | A4 | A6 | | B1 | B2 | B3 | B4 | B5 | B6 | B8 |
|---|---|---|---|---|---|---|---|---|---|---|---|---|---|
| A1 | | 0.281 | 0.903 | 0.930 | | B1 | | >.999 | >.999 | >.999 | | | |
| A2 | | | 0.606 | 0.411 | | B2 | | | >.999 | >.999 | | | |
| A3 | | | | 0.998 | | B3 | | | | >.999 | | | |
| A5 | 0.060 | 0.991 | 0.299 | 0.040* | >.999 | B7 | >.999 | >.999 | >.999 | >.999 | 0.667 | 0.191− | >.999 |

Statistical analysis of the weight gain (%) for the MPS II and WT mouse groups. P-values were calculated using the Mann-Whitney test with Bonferroni's correction. A subgroups with only IV treatment: 0.5 (A4), 2.5 (A3), 5.0 (A2), or 10 mg/kg (A1); B subgroups: each group received 0.5 (B4), 2.5 (B3), 5.0 (B2), or 10 mg/kg (B1) according to the facial SC injection dose in the IV-and-SC-treated MPS II mouse group. P-values were calculated using the Mann-Whitney test with Bonferroni's correction.

5-1. Weight Change Between Groups with/without the SC Procedure

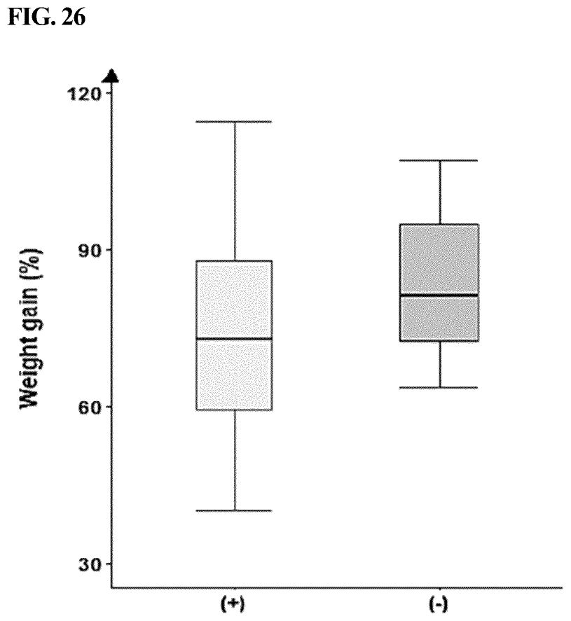

The median values of body weight gain (%) in the groups with and without the SC procedure were 75.13% (40.24-125.20%) and 81.22% (63.58-107.14%), respectively (FIG. 26). There was no statistically significant difference between the two groups (p=0.211).

### 5-2. Death Rate

Of the 70 mice at 4 weeks of age in this study, four mice died during the study, so data were collected for 66 mice at the end of the study. The comparison of the death rate between the groups without the SC procedure (A5, B7) and the groups with the SC procedure (A1, A2, A3, A4, B1, B2, B3, B4, B5, B6) showed no significant difference using Fisher's exact test (p>0.999). The statistical comparison results of the death rate by treated dose are shown in Table 11, and there was no significant difference.

TABLE 11

| | A1 | A2 | A3 | A4 | | B1 | B2 | B3 | B4 | B5 | B6 |
|---|---|---|---|---|---|---|---|---|---|---|---|
| A1 | | >.999 | >.999 | >.999 | B1 | | >.999 | — | — | | |
| A2 | | | — | 0.435 | B2 | | | >.999 | >.999 | | |
| A3 | | | | 0.435 | B3 | | | | — | | |
| A5 | >.999 | — | — | 0.435 | B7 | — | >.999 | — | — | — | — |

Statistical analysis of the death rate for IDS KO with or without the SC procedure.

P-values were calculated using Fisher's exact test using the permutation method. A subgroups with only IV treatment: 0.5 (A4), 2.5 (A3), 5.0 (A2), or 10 mg/kg (A1), B subgroups: each group received 0.5 (B4), 2.5 (B3), 5.0 (B2), or 10 (B1) according to the facial SC injection dose in the IV-and-SC-treated MPS II mouse group.

### 6. Correlation of 3D Images and Pathology

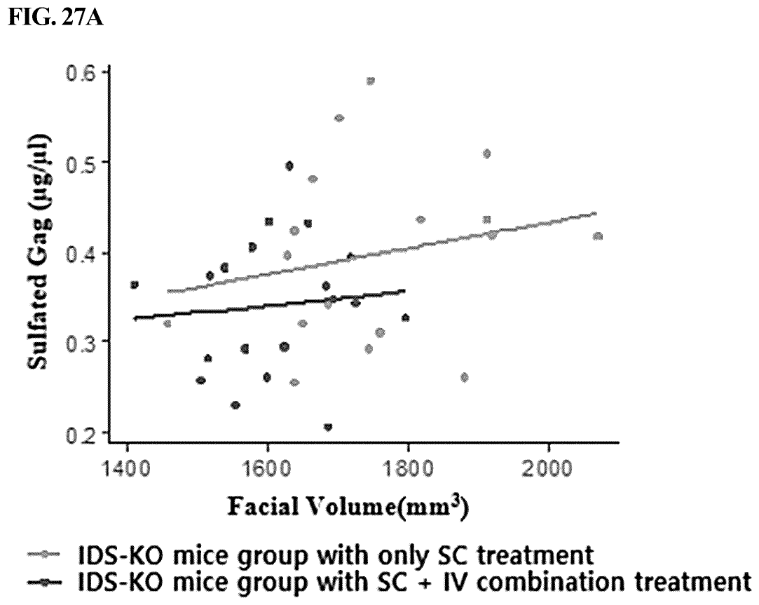

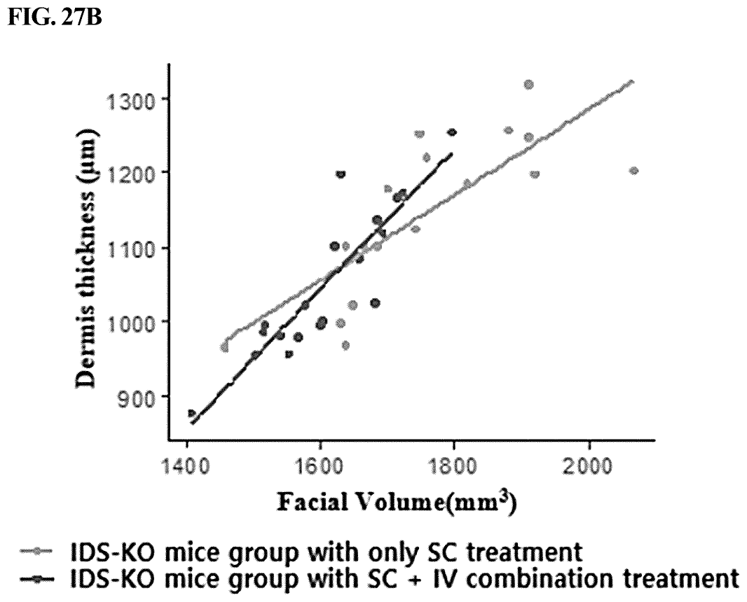

The group that underwent the SC procedure was divided into groups that only received the SC procedure (A1, A2, A3, A4) or that also received IV treatment (B1, B2, B3, B4). The correlations between the facial volume values and the s-GAG and dermis thickness values of each group were investigated. The facial volume was measured using a 3D scan before sacrificing the mice, whereas the s-GAG and dermis thickness values were measured using the biopsy tissue after animals were sacrificed. It appeared that s-GAG values were not correlated with facial volume values (A; correlation coefficient 0.22, p=0.407, B; correlation coefficient 0.03, p=0.699). There was a statistically significant correlation between dermis thickness and facial volume between the two groups (A; correlation coefficient 0.79, p=<0.001, B; correlation coefficient 0.89, p<0.001), indicating a high positive correlation in both groups (FIG. 27).

What is claimed is:

1. A method for treating of facial dysmorphism in a subject with mucopolysaccharidosis, comprising administrating a lysosomal enzyme and hyaluronic acid, which is a facial subcutaneous injection formulation.

2. The method according to claim 1, wherein the lysosomal enzyme is iduronate-2-sulfatase (IDS).

3. The method according to claim 2, wherein the iduronate-2-sulfatase comprises the amino acid sequence of SEQ ID NO: 1 or 2.

4. The method according to claim 1, wherein the lysosomal enzyme is included in a concentration of 0.5 mg/kg to 20 mg/kg.

5. The method according to claim 1, wherein the hyaluronic acid has a molecular weight of 1000 to 5000 kDa.

6. The method according to claim 1, wherein the hyaluronic acid is included at a concentration of 1 mg/ml to 15 mg/ml.

7. The method according to claim 1, wherein the mucopolysaccharidosis is type 1 or type 2 mucopolysaccharidosis.

8. The method according to claim 1, wherein the mucopolysaccharidosis is type 2 mucopolysaccharidosis.


SEQUENCE LISTING (2 sequences)

SEQ ID NO: 1 - protein, 525 aa, synthetic construct
  REGION 1..525: note = IDS-beta protein
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - protein, 525 aa, synthetic construct
  REGION 1..525: note = IDS-beta protein with cystein at positin 59 by post-translation modification
SETQANSTTDALNVLLIIVDDLRPSLGCYGDKLVRSPNIDQLASHSLLFQNAFAQQAVGAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP